(19) United States
(12) Patent Application Publication
(10) Pub. No.: US 2025/0277007 A1
(43) Pub. Date: Sep. 4, 2025

(54) CWP2 PROTEIN AS AN EFFECTIVE VACCINE AGAINST CLOSTRIDIOIDES DIFFICILE INFECTION

(71) Applicant: UNIVERSITY OF SOUTH FLORIDA, Tampa, FL (US)

(72) Inventors: Xingmin Sun, Tampa, FL (US); Shaohui Wang, Tampa, FL (US)

(21) Appl. No.: 19/066,479

(22) Filed: Feb. 28, 2025

Related U.S. Application Data
(60) Provisional application No. 63/560,005, filed on Mar. 1, 2024.
(60) Provisional application No. 63/679,343, filed on Aug. 5, 2024.

(51) Int. Cl. C07K 14/33; A61K 39/00; A61K 39/08; A61K 39/39; A61K 45/06; A61P 31/04

(52) U.S. Cl. CPC C07K 14/33; A61K 39/08; A61K 39/39; A61K 45/06; A61P 31/04; A61K 2039/55505

(57) ABSTRACT

The present disclosure relates to methods and compositions to treat or prevent Clostridioides difficile infection or recurrence.

### CROSS REFERENCE TO RELATED APPLICATIONS

This application claims the benefit of priority to U.S. Provisional Patent Application No. 63/560,005 filed on Mar. 1, 2024, and U.S. Provisional Patent Application No. 63/679,343 filed on Aug. 5, 2024, the disclosures of which are expressly incorporated by reference herein in their entireties.

### STATEMENT REGARDING FEDERALLY SPONSORED RESEARCH

This invention was made with government support under grants RO1AI132711 and 2RO1AI132711 awarded by the National Institutes of Health. The government has certain rights in the invention.

### REFERENCE TO SEQUENCE LISTING

The sequence listing submitted on Feb. 28, 2025, as an .XML entitled “11001-212US1_ST26.xml” created on Feb. 27, 2025, and having a file size of 21,318 bytes is hereby incorporated by reference pursuant to 37 C.F.R. § 1.52(e)(5).

### FIELD

Disclosed herein are methods and compositions for treating and preventing Clostridioides difficile infection.

### BACKGROUND

Clostridioides difficile (C. difficile) is a Gram-positive, spore-forming enteric pathogen responsible for a significant public health burden due to its ability to cause intestinal disorders, including inflammation and diarrhea. These symptoms primarily result from the bacterium's production of toxins, which disrupt the intestinal epithelial barrier and trigger severe inflammatory responses. C. difficile infection (CDI) is a leading cause of healthcare-associated infections globally, with increased prevalence, morbidity, and mortality rates, particularly in hospitalized and immunocompromised patients.

Standard treatment options for CDI primarily involve antibiotics such as metronidazole, vancomycin, and fidaxomicin. However, these antibiotics have significant limitations. Although they can effectively reduce the bacterial load, they also disrupt the gut microbiota, increasing the risk of recurrent infections. CDI recurrence rates remain high, with approximately 20-30% of patients experiencing a relapse after their first episode, and the risk increases with subsequent infections. Additionally, the emergence of hypervirulent C. difficile strains has further complicated treatment outcomes, contributing to higher recurrence rates, more severe disease presentations, and increased mortality.

Given the limitations of antibiotic therapies and the high recurrence rates associated with CDI, there is a critical need for alternative preventive strategies. Vaccination represents a promising approach to address these challenges by inducing protective immunity against C. difficile, thereby reducing both primary infections and recurrences. Despite efforts to develop vaccines targeting C. difficile toxins, clinical trials have yet to yield an approved vaccine. A more comprehensive approach is needed to improve vaccine efficacy.

While previous research has primarily focused on toxin-based vaccines there remains a gap in understanding other potential C. difficile based antigens as vaccine targets. There is limited information on different C. difficile strains and its potential to elicit a robust and protective immune response. Additionally, the protective efficacy of C. difficile antigen-based immunization in animal models has not been fully elucidated.

Therefore, there is a need for vaccines against CDI, especially ones that have a potential to provide long-lasting immunity against CDI, reduce recurrence rates, and alleviate the global burden of this infection. Such vaccines can complement or in some cases replace existing toxin-targeted vaccines, offering a broader and more effective preventative strategy against CDI. New methods and compositions for treating and preventing C. difficile infection are also needed. The compositions, methods, and vaccines disclosed herein address these and other needs.

### SUMMARY

Disclosed herein are methods and compositions of treating, inhibiting, reducing, decreasing, ameliorating, and/or preventing C. difficile infection in a subject in need thereof.

In some examples, disclosed herein are isolated proteins comprising a surface component of one or more strain of Clostridioides difficile (C.difficile) (such as, for example, a RT027 strain, a RT078 strain, a RT017 strain, a RT012 strain, a RT003 strain, or a RT009), wherein the surface component comprises surface proteins (such as, for example, a surface layer protein A (SlpA), a cell wall protein 84 (Cwp84), a cell wall protein 66 (Cwp66), a flagellin C protein (FliC), a flagellar capping protein D (FliD), a cysteine-rich protein CdeC, or a cysteine-rich protein CdeM), colonization factors or combination thereof. As disclosed herein the one or more strains of C.difficile is selected from a group consisting of a RT027 strain, a RT078 strain, a RT017 strain, a RT012 strain, a RT003 strain, and a RT009 strain.

In some examples, the surface component comprises a peptide from a group consisting of a surface layer protein A (SlpA), a cell wall protein 84 (Cwp84), a cell wall protein 66 (Cwp66), a flagellin C protein (FliC), a flagellar capping protein D (FliD), a cysteine-rich protein CdeC, a cysteine-rich protein CdeM, a cell wall polysaccharide PS-I, a cell wall polysaccharide PS-II, a cell wall polysaccharide PS-III, a lipoprotein CD0873, a chaperon protein DnaK, a heat shock protein GroEL and an exosporium protein BclA3.

In some examples, the surface component is a surface layer protein (SlpA) or a variant thereof.

In some examples, the surface layer protein is a cell wall protein 2 (Cwp2). As disclosed herein the Cwp2 contains an amino acid sequence SEQ ID NO: 18, or a fragment thereof. In some examples, the Cwp2 is encoded by a polynucleotide sequence SEQ ID NO: 1 or a sequence having at least 90% identity thereto.

In some examples, the Cwp2 comprises a fragment Cwp2_A, wherein the Cwp2_A comprises a sequence from about amino acid number 29 to about amino acid number 318 of SEQ ID NO: 17 or a sequence having at least 95%, 98%, 99% or 99.8% identity thereto.

In some examples, disclosed herein are immunogenic compositions comprising a C. difficile Cwp2 protein or a fragment thereof; and a pharmaceutically acceptable carrier (such as, for example, including but not limited to nanoparticle or liposome), an adjuvant (such as, for example, including but not limited to alum, aluminum hydroxide, aluminum phosphate, potassium aluminum sulfate, calcium phosphate hydroxide, Freund's complete adjuvant, Montanide®, Freund's incomplete adjuvant, iscoms, iscom matrix, ISCOMATRIX™ adjuvant, MATRIX M™ adjuvant, MATRIX C™ adjuvant, MATRIX Q™ adjuvant, AbISCO™-100 adjuvant, AbISCO™-300 adjuvant, ISCOPREP™, an ISCOPREP™ derivative, adjuvant containing ISCOPREP™ or an ISCOPREP™ derivative, QS-21, a QS-21 derivative, and an adjuvant containing QS-21 or a QS21 derivative) or a combination thereof.

In some examples, the C. difficile Cwp2 protein comprises a sequence as set forth in SEQ ID NO: 18 or a sequence having at least 95%, 98%, 99% or 99.8% identity thereto.

In some examples, the immunogenic compositions of any preceding aspect elicit at least a B cell response, a CD4+ T cell response, including Th1, Th2, or Th17, or a CD8+ T cell response.

In some examples, the immunogenic compositions of any preceding examples further comprises at least one other pharmaceutical product, wherein the at least one other pharmaceutical product is an antibiotic (such as, for example, including but not limited to metronidazole, amoxycillin, tetracycline, erythromycin, clarithromycin or tinidazole).

In some examples, disclosed herein is a method of treating, inhibiting, reducing, decreasing, ameliorating, and/or preventing C. difficile infection in a subject, comprising administering to the subject a therapeutically effective dose of a vaccine, wherein the vaccine comprises an isolated protein or a vector (such as, for example, including but not limited to a plasmid, an expression vector or a viral vector), wherein the isolated protein or the vector comprises a C.difficile Cwp2 protein or a fragment thereof.

In some examples, the vaccine of any preceding aspect further comprises a pharmaceutically acceptable carrier (such as, for example, including but not limited to nanoparticle or liposome), an adjuvant (such as, for example, including but not limited to alum, aluminum hydroxide, aluminum phosphate, potassium aluminum sulfate, calcium phosphate hydroxide, Freund's complete adjuvant, MONTANIDE™, Freund's incomplete adjuvant, iscoms, iscom matrix, ISCOMATRIX™ adjuvant, MATRIX M™ adjuvant, MATRIX C™ adjuvant, MATRIX Q™ adjuvant, AbISCO™-100 adjuvant, AbISCO™-300 adjuvant, ISCOPREP™, an ISCOPREP™ derivative, adjuvant containing ISCOPREP™ or an ISCOPREP™ derivative, QS-21, a QS-21 derivative, and an adjuvant containing QS-21 or a QS21 derivative) or a combination thereof.

In some examples, the vaccine is administered intravenously, intramuscularly, intraperitoneally, intradermally, or subcutaneously to the subject.

In some examples, the C. difficile Cwp2 protein comprises a sequence as set forth in SEQ ID NO: 18, wherein the Cwp2 protein is encoded by a polynucleotide, wherein the polynucleotide comprises a sequence as set forth in SEQ ID NO: 1 or a sequence having at least 95%, 98%, 99% or 99.8% identity thereto.

### BRIEF DESCRIPTION OF THE DRAWINGS

The accompanying figures, which are incorporated in and constitute a part of this specification, illustrate several examples described below.

FIG. 1 shows domain architecture of Cell wall protein (Cwp2) (WP_009891054.1) from C. difficile R20291. The Signal peptide (SP) is followed by the functional region, which includes Domian 1 (D1), Domain 2 (D2) and Domain 3 (D3); D2 is connected to D3 via a strand of 13 aa in D1. The Cell wall binding domain (CWB) has 3 repeated region CWB1,

CWB2 and CWB3 as indicated in UniProt. The schematic representation of the domain architecture was developed in DOG 2.0.

FIG. 2 shows predicted immunogenic regions (in yellow) of Cwp2. B cell epitopes of Cwp2 were predicted using the BepiPred-2.0 server. The residues with scores above the threshold (default value is 0.5) are predicted to be part of an epitope and colored yellow on the graph (where Y-axes depicts residue scores and X-axes residue positions in the sequence).

FIG. 3 shows Cwp2 phylogeny. Amino acid sequences of Cwp2 were aligned with the MUSCLE algorithm in MegaX before computing a maximum likelihood tree with 100 bootstrap replicates (bootstrap values >50 are displayed). Scale bars indicate 0.010 substitutions per site. The ribotype (or sequence type) of each source strain is displayed adjacent to the strain name. Ribotypes with multiple representatives have multicolored labels, while black labels indicate ribotypes with only one representative on the tree. Toxinotypes for each strain are indicated to the right of the ribotype for each strain.

FIG. 4 shows Cwp2 homology. Cwp2 sequences were aligned with MUSCLE and visualized with Jalview. The Jalview-calculated conservation scores are reported below the alignment from 0 (no conservation) to 11 (identical sequences). The ribotype of each source strain is displayed adjacent to the strain name and are color-coded for easier identification. The conserved amino acid sequences are highlighted in blue.

FIGS. 5A, 5B, 5C, 5D and 5E show expression and purification of Cwp2_A. (FIG. 5A) Cwp2_A was cloned in E. Coli BL21, and the protein was purified and analyzed on SDS-PAGE. (FIG. 5B) Immunization with Cwp2_A via the intraperitoneal (i.p.) route elicited anti-Cwp2_A antibody responses. Groups of mice (n=5 to 8) were immunized three times with 10 μg or 20 μg of Cwp2_A with aluminum. In (FIGS. 5C-5E), 20 μg of protein was used. Anti-Cwp2_A IgG/IgA titers in sera and feces were determined using an ELISA analysis. Data are presented as the mean±SEM (*p<0.05; **p<0.01; ns, not significant; 2nd and 3rd IM vs. 1st IM in (FIGS. 5C-5E)).

FIGS. 6A, 6B and 6C show immunization of mice with Cwp2_A provides protection against infection with C. difficile strain R20291. Mice were challenged with C. difficile R20291 spores (10⁶/mouse) 14 days after the third immunization of groups (n=10) with either Cwp2_A or PBS in the presence of alum. Kaplan-Meier survival plots (FIG. 6A), mean relative weight of all surviving mice (up to the day of death) (FIG. 6B), and frequency of diarrhea (FIG. 6C) are shown. Data are presented as mean relative weight±standard error (ns, not significant; *p<0.05).

FIGS. 7A, 7B and 7C show immunization of mice with Cwp2_A reduces C. difficile spore and toxin levels in the feces of mice challenged with C. difficile spores. TcdA (FIG. 7A) or TcdB (FIG. 7B) levels in feces were determined by ELISA. (FIG. 7C) R20291 spore concentrations in feces. Bars stand for means±SD. (*p<0.05, **p<0.01 versus PBS).

FIG. 8 shows an anti-Cwp2_A serum inhibits adhesion of C. difficile to HCT8 cells. The adhesion assay was performed as described in the methods. Experiments were independently repeated three times, and data are presented as mean±SEM (*p<0.05 vs. treatment with pre-immune serum).

FIGS. 9A, 9B and 9C show T cell responses. Splenocytes from immunized (n=3-4) and unimmunized (n=4) mice were isolated 13 days after the second immunization with Cwp2_A and stimulated with Cwp2_A at 10 μg/ml for 72 hours (FIGS. 9A & 9B) or 6 hours (FIG. 9C). The proliferative responses of CD4+ (FIG. 9A) and CD8+ (FIG. 9B) T cells were assayed by staining with appropriate antibodies and analyzed by flow cytometry. (FIG. 9C) IL-17, IFN-γ and TNF-α expression in the spleen cells was determined by qPCR. The y-axis value indicates the expression ratio relative to GAPDH. Data were presented as mean relative weight±standard error (N=3, *p<0.05, **p<0.01, ***p<0.001, ns, not significant).

### DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### Terminology

Terms used throughout this application are to be construed with ordinary and typical meaning to those of ordinary skill in the art. However, Applicant desires that the following terms be given the particular definition as defined below.

As used herein, the article “a,” “an,” and “the” means “at least one,” unless the context in which the article is used clearly indicates otherwise.

“Administration” to a subject or “administering” includes any route of introducing or delivering to a subject an agent. Administration can be carried out by any suitable route, including oral, intravenous, intraperitoneal, intranasal, inhalation and the like. Administration includes self-administration and the administration by another.

The terms “about” and “approximately” are defined as being “'close to” as understood by one of ordinary skill in the art. In one non-limiting embodiment, the terms are defined to be within 10%. In another non-limiting embodiment, the terms are defined to be within 5%. In still another non-limiting embodiment, the terms are defined to be within 1%.

The term “cancer” or “neoplasms” used herein meant to include all types of cancerous growths or oncogenic processes, metastatic tissues or malignantly transformed cells, tissues, or organs, irrespective of histopathologic type or stage of invasiveness. The terms “cancer” or “neoplasms” include malignancies of the various organ systems, such as malignancies affecting skin, brain, spinal cord, cervix, bladder, lung, breast, thyroid, lymphoid tissues, connecting tissues, gastrointestinal, and genito-urinary tracts, that include, but are not limited to, glioma, melanoma, lung cancer, breast cancer, cervical squamous cell carcinoma, bladder cancer, and soft tissue sarcoma. The term “cancer metastasis” has its general meaning in the art and refers to the spread of a tumor from one organ or part to another non-adjacent organ or part.

The term “comprising” and variations thereof as used herein is used synonymously with the term “including” and variations thereof and are open, non-limiting terms. Although the terms “comprising” and “including” have been used herein to describe various examples, the terms “consisting essentially of” and “consisting of” can be used in place of “comprising” and “including” to provide for more specific examples and are also disclosed.

A “composition” is intended to include a combination of active agent and another compound or composition, inert (for example, a detectable agent or label) or active, such as an adjuvant.

As used herein, the terms “determining,” “measuring,” and “assessing,” and “assaying” are used interchangeably and include both quantitative and qualitative determinations.

By the term “effective amount” of a therapeutic agent is meant a nontoxic but sufficient amount of a beneficial agent to provide the desired effect. The amount of beneficial agent that is “effective” will vary from subject to subject, depending on the age and general condition of the subject, the particular beneficial agent or agents, and the like. Thus, it is not always possible to specify an exact “effective amount.” However, an appropriate “effective” amount in any subject case may be determined by one of ordinary skill in the art using routine experimentation. Also, as used herein, and unless specifically stated otherwise, an “effective amount” of a beneficial can also refer to an amount covering both therapeutically effective amounts and prophylactically effective amounts.

An “effective amount” of a drug necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

As used herein the term “encoding” refers to the inherent property of specific sequences of nucleotides in a nucleic acid, to serve as templates for synthesis of other molecules having a defined sequence of nucleotides (i.e. rRNA, tRNA, other RNA molecules) or amino acids and the biological properties resulting therefrom.

The “fragments” or “functional fragments,” whether attached to other sequences or not, can include insertions, deletions, substitutions, or other selected modifications of particular regions or specific amino acids residues, provided the activity of the fragment is not significantly altered or impaired compared to the nonmodified peptide or protein. These modifications can provide for some additional property, such as to remove or add amino acids capable of disulfide bonding, to increase its bio-longevity, to alter its secretory characteristics, etc. In any case, the functional fragment must possess a bioactive property, such as antigen binding and antigen recognition.

The term “gene” or “gene sequence” refers to the coding sequence or control sequence, or fragments thereof. A gene may include any combination of coding sequence and control sequence, or fragments thereof. Thus, a “gene” as referred to herein may be all or part of a native gene. A polynucleotide sequence as referred to herein may be used interchangeably with the term “gene”, or may include any coding sequence, non-coding sequence or control sequence, fragments thereof, and combinations thereof. The term “gene” or “gene sequence” includes, for example, control sequences upstream of the coding sequence (for example, the ribosome binding site).

The term “isolating” as used herein refers to isolation from a biological sample, i.e., blood, plasma, tissues, exosomes, or cells. As used herein the term “isolated,” when used in the context of, e.g., a nucleic acid, refers to a nucleic acid of interest that is at least 60% free, at least 75% free, at least 90% free, at least 95% free, at least 98% free, and even at least 99% free from other components with which the nucleic acid is associated with prior to purification.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

The term “nucleic acid” refers to a natural or synthetic molecule comprising a single nucleotide or two or more nucleotides linked by a phosphate group at the 3′ position of one nucleotide to the 5′ end of another nucleotide. The nucleic acid is not limited by length, and thus the nucleic acid can include deoxyribonucleic acid (DNA) or ribonucleic acid (RNA).

The term “oligonucleotide” denotes single- or double-stranded nucleotide multimers of from about 2 to up to about 100 nucleotides in length. Suitable oligonucleotides may be prepared by the phosphoramidite method described by Beaucage and Carruthers, Tetrahedron Lett., 22: 1859-1862 (1981), or by the triester method according to Matteucci, et al., J. Am. Chem. Soc., 103:3185 (1981), both incorporated herein by reference, or by other chemical methods using either a commercial automated oligonucleotide synthesizer or VLSIPS™ technology. When oligonucleotides are referred to as “double-stranded,” it is understood by those of skill in the art that a pair of oligonucleotides exist in a hydrogen-bonded, helical array typically associated with, for example, DNA. In addition to the 100% complementary form of double-stranded oligonucleotides, the term “double-stranded,” as used herein is also meant to refer to those forms which include such structural features as bulges and loops, described more fully in such biochemistry texts as Stryer, Biochemistry, Third Ed., (1988), incorporated herein by reference for all purposes.

The term “polynucleotide” refers to a single or double stranded polymer composed of nucleotide monomers.

The term “polypeptide” refers to a compound made up of a single chain of D- or L-amino acids or a mixture of D- and L-amino acids joined by peptide bonds.

The terms “peptide,” “protein,” and “polypeptide” are used interchangeably to refer to a natural or synthetic molecule comprising two or more amino acids linked by the carboxyl group of one amino acid to the alpha amino group of another.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably over a region that is 10-50 amino acids or 20-50 nucleotides in length. As used herein, percent (%) nucleotide sequence identity is defined as the percentage of amino acids in a candidate sequence that are identical to the nucleotides in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

One example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990) J. Mol. Biol. 215:403-410). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01.

As used herein, the term “pharmaceutically acceptable” component can refer to a component that is not biologically or otherwise undesirable, i.e., the component may be incorporated into a pharmaceutical formulation of the invention and administered to a subject as described herein without causing any significant undesirable biological effects or interacting in a deleterious manner with any of the other components of the formulation in which it is contained. When the term “pharmaceutically acceptable” is used to refer to an excipient, it is generally implied that the component has met the required standards of toxicological and manufacturing testing or that it is included on the Inactive Ingredient Guide prepared by the U.S. Food and Drug Administration.

The term “subject” or “host” refers to any individual who is the target of administration or treatment. The subject can be a vertebrate, for example, a mammal. Thus, the subject can be a human or veterinary patient. The term “patient” refers to a subject under the treatment of a clinician, e.g., physician. The subject can be either male or female.

A control sample or a reference sample as described herein can be a sample from a healthy subject or sample, a wild-type subject or sample, or from populations thereof. A reference value can be used in place of a control or reference sample, which was previously obtained from a healthy subject or a group of healthy subjects or a wild-type subject or sample. A control sample or a reference sample can also be a sample with a known amount of a detectable compound or a spiked sample.

The term “tissue” refers to a group or layer of similarly specialized cells which together perform certain special functions. The term “tissue” is intended to include, blood, blood preparations such as plasma and serum, bones, joints, muscles, smooth muscles, lung tissues, and organs.

As used herein, the terms “treating” or “treatment” of a subject includes the administration of a drug to a subject with the purpose of preventing, curing, healing, alleviating, relieving, altering, remedying, ameliorating, improving, stabilizing or affecting a disease or disorder (e.g., a cancer), or a symptom of a disease or disorder. The terms “treating” and “treatment” can also refer to reduction in severity and/or frequency of symptoms, elimination of symptoms and/or underlying cause, prevention of the occurrence of symptoms and/or their underlying cause, and improvement or remediation of damage.

As used herein, a “therapeutically effective amount” of a therapeutic agent refers to an amount that is effective to achieve a desired therapeutic result, and a “prophylactically effective amount” of a therapeutic agent refers to an amount that is effective to prevent an unwanted physiological condition (e.g. cancer). Therapeutically effective and prophylactically effective amounts of a given therapeutic agent will typically vary with respect to factors such as the type and severity of the disorder or disease being treated and the age, gender, and weight of the subject.

The term “therapeutically effective amount” can also refer to an amount of a therapeutic agent, or a rate of delivery of a therapeutic agent (e.g., amount over time), effective to facilitate a desired therapeutic effect. The precise desired therapeutic effect will vary according to the condition to be treated, the tolerance of the subject, the drug and/or drug formulation to be administered (e.g., the potency of the therapeutic agent (drug), the concentration of drug in the formulation, and the like), and a variety of other factors that are appreciated by those of ordinary skill in the art.

In some examples, numbers expressing quantities of ingredients, properties such as molecular weight, reaction conditions, and so forth, used to describe and claim certain examples of the present disclosure are to be understood as being modified in some instances by the term “about.” In some examples, the term “about” is used to indicate that a value includes the standard deviation of the mean for the device or method being employed to determine the value. In some examples, the numerical parameters set forth in the written description and attached claims are approximations that can vary depending upon the desired properties sought to be obtained by a particular embodiment. In some examples, the numerical parameters should be construed in light of the number of reported significant digits and by applying ordinary rounding techniques. Notwithstanding that the numerical ranges and parameters setting forth the broad scope of some examples of the present disclosure are approximations, the numerical values set forth in the specific examples are reported as precisely as practicable. The numerical values presented in some examples of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

Throughout this application, various publications are referenced. The disclosures of these publications in their entirety are hereby incorporated by reference into this application in order to more fully describe the state of the art to which this pertains. The references disclosed are also individually and specifically incorporated by reference herein for the material contained in them that is discussed in the sentence in which the reference is relied upon.

### Molecular Engineering

The term “transfection,” as used herein, refers to the process of introducing nucleic acids into cells by non-viral methods. The term “transduction,” as used herein, refers to the process whereby foreign DNA is introduced into another cell via a viral vector.

The terms “heterologous DNA sequence”, “exogenous DNA segment”, or “heterologous nucleic acid,” as used herein, each refers to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling or cloning. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

Expression vector, expression construct, plasmid, or recombinant DNA construct is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

An “expression vector,” otherwise known as an “expression construct,” is generally a plasmid or virus designed for gene expression in cells. The vector is used to introduce a specific gene into a target cell, and can commandeer the cell's mechanism for protein synthesis to produce the protein encoded by the gene. Expression vectors are the basic tools in biotechnology for the production of proteins. The vector is engineered to contain regulatory sequences that act as enhancer and/or promoter regions and lead to efficient transcription of the gene carried on the expression vector. The goal of a well-designed expression vector is the efficient production of protein, and this may be achieved by the production of significant amount of stable messenger RNA, which can then be translated into protein. The expression of a protein may be tightly controlled, and the protein is only produced in significant quantity, when necessary, through the use of an inducer, in some systems however the protein may be expressed constitutively. As described herein, Escherichia coli is used as the host for protein production, but other cell types may also be used.

In molecular biology, an “inducer” is a molecule that regulates gene expression. An inducer can function in two ways, such as:
(i) By disabling repressors. The gene is expressed because an inducer binds to the repressor. The binding of the inducer to the repressor prevents the repressor from binding to the operator. RNA polymerase can then begin to transcribe operon genes.
(ii) By binding to activators. Activators generally bind poorly to activator DNA sequences unless an inducer is present. An activator binds to an inducer and the complex binds to the activation sequence and activates target gene. Removing the inducer stops transcription. Because a small inducer molecule is required, the increased expression of the target gene is called induction.

Repressor proteins bind to the DNA strand and prevent RNA polymerase from being able to attach to the DNA and synthesize mRNA. Inducers bind to repressors, causing them to change shape and preventing them from binding to DNA. Therefore, they allow transcription, and thus gene expression, to take place.

For a gene to be expressed, its DNA sequence must be copied (in a process known as transcription) to make a smaller, mobile molecule called messenger RNA (mRNA), which carries the instructions for making a protein to the site where the protein is manufactured (in a process known as translation). Many different types of proteins can affect the level of gene expression by promoting or preventing transcription. In prokaryotes (such as bacteria), these proteins often act on a portion of DNA known as the operator at the beginning of the gene. The promoter is where RNA polymerase, the enzyme that copies the genetic sequence and synthesizes the mRNA, attaches to the DNA strand.

Some genes are modulated by activators, which have the opposite effect on gene expression as repressors. Inducers can also bind to activator proteins, allowing them to bind to the operator DNA where they promote RNA transcription. Ligands that bind to deactivate activator proteins are not, in the technical sense, classified as inducers, since they have the effect of preventing transcription.

A “promoter” is generally understood as a nucleic acid control sequence that directs transcription of a nucleic acid. An inducible promoter is generally understood as a promoter that mediates transcription of an operably linked gene in response to a particular stimulus. A promoter can include necessary nucleic acid sequences near the start site of transcription, such as, in the case of a polymerase II type promoter, a TATA element. A promoter can optionally include distal enhancer or repressor elements, which can be located as much as several thousand base pairs from the start site of transcription.

A “ribosome binding site” or “(RBS)” refers to a sequence of nucleotides upstream of the start codon of an mRNA transcript that is responsible for the recruitment of a ribosome during the initiation of translation. Generally, RBS refers to bacterial sequences, although internal ribosome entry sites (IRES) have been described in mRNAs of eukaryotic cells or viruses that infect eukaryotes. Ribosome recruitment in eukaryotes is generally mediated by the 5′ cap present on eukaryotic mRNAs.

A “transcribable nucleic acid molecule” as used herein refers to any nucleic acid molecule capable of being transcribed into an RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit translation of a specific RNA molecule of interest. For the practice of the present disclosure, conventional compositions and methods for preparing and using constructs and host cells are well known to one skilled in the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754).

The “transcription start site” or “initiation site” is the position surrounding the first nucleotide that is part of the transcribed sequence, which is also defined as position +1. With respect to this site all other sequences of the gene and its controlling regions can be numbered. Downstream sequences (i.e., further protein encoding sequences in the 3′ direction) can be denominated positive, while upstream sequences (mostly of the controlling regions in the 5′ direction) are denominated negative.

“Operably linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A construct of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism such as a bacterium, cyanobacterium, animal, or a plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include, but are not limited to, methods using paired primers, nested primers, single specific primers, degenerate primers, gene-specific primers, vector-specific primers, partially mismatched primers, and the like. The term “untransformed” refers to normal cells that have not been through the transformation process.

“Wild-type” refers to a virus or organism found in nature without any known mutation. Design, generation, and testing of the variant nucleotides, and their encoded polypeptides, having the above-required percent identities and retaining a required activity of the expressed protein is within the skill of the art. For example, directed evolution and rapid isolation of mutants can be according to methods described in references including, but not limited to, Link et al. (2007) Nature Reviews 5(9), 680-688; Sanger et al. (1991) Gene 97(1), 119-123; Ghadessy et al. (2001) Proc Natl Acad Sci USA 98(8) 4552-4557. Thus, one skilled in the art could generate a large number of nucleotide and/or polypeptide variants having, for example, at least 95-99% identity to the reference sequence described herein and screen such for desired phenotypes according to methods routine in the art.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2, or Megalign (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=XN100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A. For example, the percent identity can be at least 80% or about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%.

Substitution refers to the replacement of one amino acid with another amino acid in a protein or the replacement of one nucleotide with another in DNA or RNA. Insertion refers to the insertion of one or more amino acids in a protein or the insertion of one or more nucleotides with another in DNA or RNA. Deletion refers to the deletion of one or more amino acids in a protein or the deletion of one or more nucleotides with another in DNA or RNA. Generally, substitutions, insertions, or deletions can be made at any position so long as the required activity is retained. So-called conservative exchanges can be carried out in which the amino acid which is replaced has a similar property as the original amino acid, for example, the exchange of Glu by Asp, Gln by Asn, Val by Ile, Leu by Ile, and Ser by Thr. For example, amino acids with similar properties can be Aliphatic amino acids (e.g., Glycine, Alanine, Valine, Leucine, Isoleucine); hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. An amino acid sequence can be modulated with the help of art-known computer simulation programs that can produce a polypeptide with, for example, improved activity or altered regulation. On the basis of these artificially generated polypeptide sequences, a corresponding nucleic acid molecule coding for such a modulated polypeptide can be synthesized in-vitro using the specific codon-usage of the desired host cell.

Host cells can be transformed using a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transformed cells can be selected and propagated to provide recombinant host cells that comprise the expression vector stably integrated in the host cell genome.

Exemplary nucleic acids that may be introduced to a host cell include, for example, DNA sequences or genes from another species, or even genes or sequences which originate with or are present in the same species, but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA that is already present in the cell, DNA from another individual of the same type of organism, DNA from a different organism, or a DNA generated externally, such as a DNA sequence containing an antisense message of a gene, or a DNA sequence encoding a synthetic or modified version of a gene.

Methods of down-regulation or silence genes are known in art. For example, expressed protein activity can be downregulated or eliminated using antisense oligonucleotides (ASOs), protein aptamers, nucleotide aptamers, and RNA interference (RNAi) (e.g., small interfering RNAs (siRNA), short hairpin RNA (shRNA), and micro RNAs (miRNA) (see e.g., Rinaldi and Wood (2017) Nature Reviews Neurology 14, describing ASO therapies; Fanning and Symonds (2006) Handb Exp Pharmacol. 173, 289-303G, describing hammerhead ribozymes and small hairpin RNA; Helene, et al. (1992) Ann. N.Y. Acad. Sci. 660, 27-36; Maher (1992) Bioassays 14(12): 807-15, describing targeting deoxyribonucleotide sequences; Lee et al. (2006) Curr Opin Chem Biol. 10, 1-8, describing aptamers; Reynolds et al. (2004) Nature Biotechnology 22(3), 326-330, describing RNAi; Pushparaj and Melendez (2006) Clinical and Experimental Pharmacology and Physiology 33(5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, TX; Sigma Aldrich, MO; Invitrogen). Several siRNA molecule design programs using a variety of algorithms are known to the art (see e.g., Cenix algorithm, Ambion; BLOCK-iT™ RNAi Designer, Invitrogen; siRNA Whitehead Institute Design Tools, Bioinformatics & Research Computing). Traits influential in defining optimal siRNA sequences include G/C content at the termini of the siRNAs, Tm of specific internal domains of the siRNA, siRNA length, position of the target sequence within the CDS (coding region), and nucleotide content of the 3′ overhangs.

As would be apparent, the sequencing may be done using a next generation sequencing platform, e.g., Illumina's reversible terminator method, Roche's pyrosequencing method, Life Technologies' sequencing by ligation (the SOLiD platform) or Life Technologies' Ion Torrent platform, etc. Examples of such methods are described in the following references: Margulies et al (Nature 2005 437: 376-80); Ronaghi et al (Analytical Biochemistry 1996 242: 84-9); Shendure (Science 2005 309: 1728); Imelfort et al (Brief Bioinform. 2009 10:609-18); Fox et al (Methods Mol Biol. 2009; 553:79-108); Appleby et al (Methods Mol Biol. 2009; 513:19-39) and Morozova (Genomics. 2008 92:255-64), which are incorporated by reference for the general descriptions of the methods and the particular steps of the methods, including all starting products, reagents, and final products for each of the steps. In other examples, the sequencing may be done using nanopore sequencing (e.g. as described in Soni et al Clin Chem 53: 1996-2001 2007, or as described by Oxford Nanopore Technologies).

### Compositions

Clostridium difficile is the primary causative agent of antibiotic associated diarrhea. Increasing resistance to antibiotics in recent decades has resulted in a reduction in the efficacy of standard methods of treatment. This presents a clear need for a greater understanding of the bacterium, so that alternative methods of treating C. difficile infection (CDI) may be developed.

Clostridioides difficile (C.difficile) infection (CDI) is a bacterial infectious disease of the gastrointestinal tract caused by Clostridium difficile (C. difficile), a toxin-producing Gram-positive anaerobic, spore-forming bacillus. As used herein, CDI includes recurrent CDI, which is defined as complete resolution of CDI while on appropriate therapy, followed by recurrence of CDI after treatment has been stopped. CDI is often associated with disorders of the gastrointestinal tract such as dysbiosis, Crohn's disease, ulcerative colitis, enteritis, irritable bowel syndrome, inflammatory bowel disease, diarrhea, antibiotic-associated diarrhea, and diverticular disease. In some examples, there are provided compositions and methods for prevention or treatment of disorders of the gastrointestinal tract associated with CDI such as, without limitation, dysbiosis, Crohn's disease, ulcerative colitis, enteritis, irritable bowel syndrome, inflammatory bowel disease, diarrhea, antibiotic-associated diarrhea, and diverticular disease.

In some examples, disclosed herein is an isolated protein comprising a surface component of one or more strain of Clostridioides difficile (C.difficile), wherein the surface component comprises surface proteins, colonization factors or a combination thereof. As disclosed herein, in some examples, the one or more strains of C.difficile include but not limited to a RT027 strain, a RT078 strain, a RT017 strain, a RT012 strain, a RT003 strain, and a RT009 strain. As used herein, the term “isolated” refers to a molecule that by virtue of its origin or source of derivation (1) is not associated with naturally associated components that accompany it in its native state, (2) is free of other macromolecules (e.g., proteins, glycans) from the same species, (3) is expressed by a cell from a different species, or (4) does not occur in nature. Thus, a protein or peptide that is chemically synthesized or synthesized in a cellular system different from the cell from which it naturally originates will be “isolated” from its naturally associated components. A protein or peptide may also be rendered substantially free of naturally associated components by isolation, using purification or separation techniques well known in the art. Surface components from C. difficile used in compositions and methods described herein are generally provided in purified or substantially purified form, i.e., substantially free from other glycopeptides and polypeptides, particularly from host cell proteins or polypeptides. In some examples, the isolated protein is at least about 50% pure, at least about 60% pure, at least about 70% pure, at least about 80%, at least about 90% pure, or at least about 95% pure (by weight).

As disclosed herein, in some examples, the surface component comprise a peptide which includes but is not limited to surface layer protein A (SlpA), cell wall protein 84 (Cwp84), cell wall protein 66 (Cwp66), flagellin C protein (FliC), flagellar capping protein D (FliD), cysteine-rich protein CdeC, cysteine-rich protein CdeM, cell wall polysaccharide PS-I, cell wall polysaccharide PS-II, cell wall polysaccharide PS-III, lipoprotein CD0873, chaperon protein DnaK, heat shock protein GroEL or exosporium protein BclA3.

As disclosed herein, in some examples, the surface component peptide includes but is not limited to a surface layer protein (SlpA) or a variant thereof. As used herein, the term “SlpA” refers to the major surface-layer protein (S-layer protein A) of Clostridioides difficile, which forms a crystalline, paracrystalline, or amorphous layer on the bacterial cell surface. SlpA is processed post-translationally to generate two subunits: a high-molecular-weight (HMW) and low-molecular-weight (LMW) component, which assemble to form the protective S-layer structure. This protein plays a crucial role in bacterial adhesion, immune evasion, and interaction with host cells. Given its surface exposure and immunogenicity, SlpA is considered a promising vaccine candidate and diagnostic target for C. difficile infections. Additionally, SlpA exhibits significant sequence variability among C. difficile strains, contributing to differences in virulence and host immune recognition.

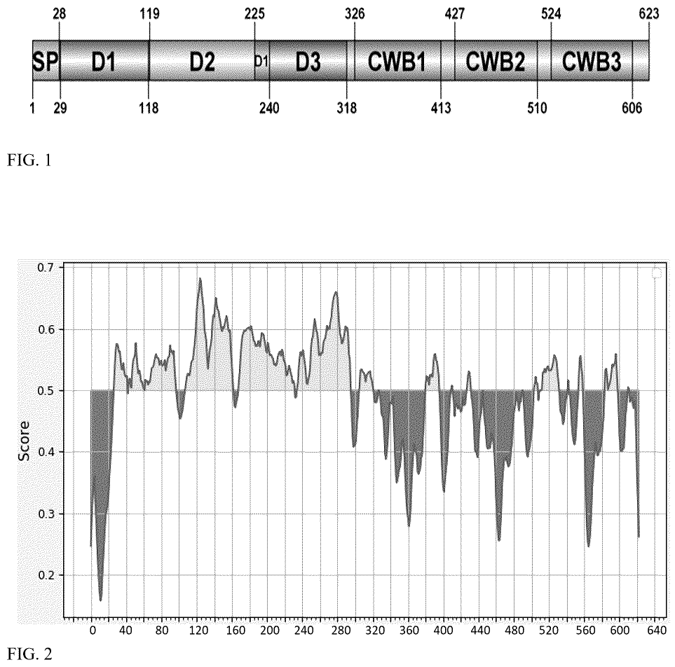

As disclosed herein, in some examples, the surface layer protein includes but is not limited to a cell wall protein 2 (Cwp2). Also disclosed herein, in some examples, the Cwp2 contains an amino acid sequence SEQ ID NO: 18, a derivative, a fragment, a mutant or a variant thereof. In some examples, the Cwp2 is encoded by a polynucleotide sequence SEQ ID NO: 1 or a sequence having at least 80%, 85%, 87%, 89%, 90%, 91%, 92%, 93%, 94%, 95%, 96%, 97%, 98%, 99% or 100% identity thereto. A “variant,” “mutant,” or “derivative” of a particular nucleic acid sequence may be defined as a nucleic acid sequence having at least 50% sequence identity to the particular nucleic acid sequence over a certain length of one of the nucleic acid sequences using blastn with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). In some examples a variant polynucleotide may show, for example, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length relative to a reference polynucleotide. The cwp2 gene is found within the slpA locus. As used herein, in FIG. 1, the term “Cwp2” (Cell Wall Protein 2) refers to a structural and functional protein of Clostridioides difficile that comprises multiple domains essential for its role in bacterial surface layer (S-layer) assembly, cell wall anchoring, and host-pathogen interactions. The protein includes an N-terminal signal peptide (SP, residues 1-28) that directs secretion through the Sec-dependent pathway and is cleaved post-translationally to facilitate proper localization. Following the signal sequence, the protein contains Domain 1 (D1, residues 29-118), Domain 2 (D2, residues 119-224), and Domain 3 (D3, residues 225-318), which may contribute to structural integrity, adhesion properties, and interactions with host cells or other bacterial proteins. These domains may also include immunogenic regions capable of eliciting an immune response. The C-terminal region consists of three cell wall binding (CWB) domains: CWB1 (residues 326-413), CWB2 (residues 427-510), and CWB3 (residues 524-606), which mediate the attachment of Cwp2 to the peptidoglycan layer of the bacterial cell wall, ensuring its stable incorporation into the S-layer. The modular architecture of Cwp2 facilitates its role in bacterial adherence, immune evasion, and structural stabilization, making it a viable target for immunogenic compositions, therapeutic interventions, or antimicrobial strategies aimed at preventing or treating C. difficile infections.

As disclosed herein, in some examples, the Cwp2 comprises a fragment which includes but is not limited to Cwp2_A. As disclosed herein, in some examples the Cwp2_A comprises a sequence from about amino acid number 29 to about amino acid number 318 of SEQ ID NO: 17 or a sequence having at least 95%, 98%, 99% or 99.8% identity thereto. In some examples the Cwp2_A comprises a sequence from about amino acid number 15, 16, 17, 18, 19, 20, 21, 22, 23, 24, 25, 26, 27, 28, 29, 30, 31, 32, 33, 34, 35, 36, 37, 38, 39, 40 to about amino acid number 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340 of SEQ ID NO: 18. Colonization of the gut by Clostridium difficile requires the adhesion of the bacterium to host cells. A range of cell surface located factors have been linked to adhesion including the S-layer protein LMW. The S-layer of C. difficile may contain many proteins including Cwp2. As demonstrated in Example 6, lack of Cwp2 increases TcdA (toxin A) release and impairs cellular adherence in vitro. Cwp2 A is the ‘functional’ region of Cwp2, consisting of residues 29-318 at 1.9 Å. The adhesive properties of Cwp2 are predicted to be mediated by the variable loop regions in domain 2. As used herein, the term “Cwp2_A” refers to a specific functional variant, domain, or truncated form of the Clostridioides difficile Cell Wall Protein 2 (Cwp2) that retains key structural and immunogenic properties associated with bacterial S-layer formation, host-pathogen interactions, and cell wall anchoring. Cwp2_A may comprise one or more domains of the full-length Cwp2 protein, including but not limited to structural domains (D1, D2, D3) involved in protein stability and bacterial adhesion, and cell wall binding domains (CWB1, CWB2, CWB3) responsible for anchoring to the peptidoglycan layer. The Cwp2_A variant may be naturally occurring or engineered to enhance antigenicity, stability, or immunogenic potential for use in immunogenic compositions, vaccines, diagnostics, or therapeutic interventions against C. difficile infection. Additionally, Cwp2_A may include modified sequences that optimize expression, folding, or immune recognition, making it a candidate for recombinant protein-based therapeutics or antibody-targeting approaches.

As used herein, the term “antigen” refers to a substance that prompts the generation of antibodies and can cause an immune response. The terms “antigen” and “immunogen” are used interchangeably herein, although, in a strict sense, immunogens are substances that elicit a response from the immune system, whereas antigens are defined as substances that bind to specific antibodies. An antigen or fragment thereof can be a molecule (i.e., an epitope) that makes contact with a particular antibody. When Cwp2 or a fragment thereof is used to immunize a host animal, numerous regions of the Cwp2 can induce the production of antibodies (i.e., elicit the immune response), which bind specifically to the antigen.

In some examples, “variants”, “analogs”, and “fragments” of Cwp2 refers to an amino acid sequence of the naturally occurring protein or peptide in which a small number of amino acids have been substituted, inserted, or deleted, and which retains the relevant biological activity or function of the starting protein. For example, in the case of an antigen for use in a vaccine, a variant may retain the immunogenic characteristics of the starting protein, sufficient for its intended use in inducing immunity. In the case of an antibody, a variant may retain the antigen-binding properties of the starting protein, sufficient for its intended use in binding specifically to antigen.

In some examples, a variant includes one or more conservative amino acid substitutions, one or more non-conservative amino acid substitutions, one or more deletions, and/or one or more insertions. A conservative substitution is one in which an amino acid residue is substituted by another amino acid residue having similar characteristics (e.g., charge or hydrophobicity). In general, a conservative amino acid substitution will not substantially change the functional properties of a protein. Examples of groups of amino acids that have side chains with similar chemical properties include: 1) aliphatic side chains: glycine, alanine, valine, leucine, and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartic acid and glutamic acid; and 7) sulfur-containing side chains: cysteine and methionine. Exemplary conservative amino acids substitution groups are valine-leucine-isoleucine, phenylalanine-tyrosine, lysine-arginine, alanine-valine, glutamate-aspartate, and asparagine-glutamine. Other conservative amino acid substitutions are known in the art and are included herein. Non-conservative substitutions, such as replacing a basic amino acid with a hydrophobic one, are also well-known in the art.

As used herein, an “analog” refers to an amino acid sequence of the naturally occurring protein in which one or more amino acids have been replaced by amino acid analogs. Non-limiting examples of amino acid analogs include non-naturally occurring amino acids, synthetic amino acids, amino acids which only occur naturally in an unrelated biological system, modified amino acids from mammalian systems, polypeptides with substituted linkages, as well as other modifications known in the art, both naturally occurring and non-naturally occurring. In some examples, analogs include modifications which increase glycoprotein or glycopeptide stability. In one embodiment, an analog includes a beta amino acid, a gamma amino acid, or a D-amino acid.

A “fragment” refers to a portion of the starting molecule which retains the relevant biological activity or function (e.g, antigenicity, antigen-binding, immunogenicity) of the starting molecule.

A “biologically active” or “functional fragment”, fragment, variant, or analog generally retains biological activity or function of the starting molecule, sufficient for use in the present compositions and methods. Thus, a “biologically active” or “functional fragment”, fragment, variant, or analog may retain the binding specificity, the antigenicity, or the immunogenicity of the starting molecule. In some examples, a fragment, variant or analog has at least about 70%, at least about 80%, at least about 85%, at least about 90%, at least about 95%, or at least about 98% sequence identity to the starting molecule (e.g., protein).

Variants, fragments, or analogs may also be modified at the N- and/or C-terminal ends to allow the polypeptide or fragment to be conformationally constrained and/or to allow coupling to a pharmaceutically acceptable carrier or an adjuvant. In some examples, disclosed herein are immunogenic compositions comprising a C. difficile Cwp2 protein or a fragment thereof; and a pharmaceutically acceptable carrier, an adjuvant or a combination thereof. In some examples, the pharmaceutically acceptable carrier includes but is not limited to oil-in-water emulsions (e.g., MF59, AS03, AF03), TLR agonists (e.g., CpG oligodeoxynucleotides, monophosphoryl lipid A), saponin-based adjuvants (e.g., QS-21), cytokines (e.g., GM-CSF, IL-2, IL-12), lipid-based carriers like liposomes, virosomes, and lipid nanoparticles (LNPs), polymer-based carriers such as PLGA nanoparticles, chitosan nanoparticles, and PEGylated polymers, viral vectors including adenovirus, lentivirus, and vesicular stomatitis virus (VSV), as well as stabilizers and preservatives like sucrose, trehalose, lactose, gelatin, albumin, thimerosal, and buffers such as phosphate-buffered saline (PBS) and Tris buffer, nanoparticle or liposome.

Adjuvants generally increase the specificity and/or the level of immune response. An adjuvant may thus reduce the quantity of antigen necessary to induce an immune response, and/or the frequency of injection necessary in order to generate a sufficient immune response to benefit the subject. Any compound or compounds that act to increase an immune response to an antigen and are suitable for use in a subject (e.g., pharmaceutically acceptable) may be used as an adjuvant in compositions, vaccines, and methods of the invention. In some examples, the adjuvant includes but not limited to alum, aluminum hydroxide, aluminum phosphate, potassium aluminum sulfate, calcium phosphate hydroxide, Freund's complete adjuvant, MONTANIDE™, Freund's incomplete adjuvant, iscoms, iscom matrix, ISCOMATRIX™ adjuvant, MATRIX M™ adjuvant, MATRIX C™ adjuvant, MATRIX Q™ adjuvant, AbISCO™-100 adjuvant, AbISCO™-300 adjuvant, ISCOPREP™, an ISCOPREP™ derivative, adjuvant containing ISCOPREP™ or an ISCOPREP™ derivative, QS-21, a QS-21 derivative, and an adjuvant containing QS-21 or a QS21 derivative.

As disclosed herein, in some examples, the C. difficile Cwp2 protein comprises a sequence as set forth in SEQ ID NO: 18 or a sequence having at least 95%, 98%, 99% or 99.8% identity thereto.

As disclosed herein, in some examples, the immunogenic compositions of any preceding aspect elicit an immune response include but not limited to at least a B cell response, a CD4+ T cell response, or a CD8+ T cell response. As used herein, the term “immune response” refers to the response of immune system cells to external or internal stimuli (e.g., antigens, cell surface receptors, cytokines, chemokines, and other cells) producing biochemical changes in the immune cells that result in immune cell migration, killing of target cells, phagocytosis, production of antibodies, production of soluble effectors of the immune response, and the like. An “immunogenic” molecule is one that is capable of producing an immune response in a subject after administration. As disclosed herein, in some examples, the CD4+ T cell response includes but is not limited to Th1, Th2, or Th17 response. In some examples, a Th1 response is associated with the production of cytokines such as interferon gamma (IFN-γ), interleukin-2 (IL-2), and tumor necrosis factor alpha (TNF-α); a Th2 response is associated with IL-4, IL-5, and IL-13; and a Th17 response is associated with IL-17, IL-21, and IL-22. By tailoring the immunogenic composition to favor one or more of these cytokine profiles, it is possible to optimize the nature and magnitude of the immune response, thereby enhancing protection against or clearance of the CDI.

As disclosed herein, in some examples, the immunogenic compositions of any preceding aspect, further comprises at least one other pharmaceutical product. In some examples, the at least one other pharmaceutical product includes but is not limited to standard antibiotics, fecal microbial transplantation, monoclonal antibodies (e.g., Bezlotoxumab and Actoxumab), SER-109 and RBX2660 (live biotherapeutic products derived from fecal microbiota), rifaximin, probiotics, immunotherapeutic products targeting toxins, or proton pump inhibitors. In some examples, the antibiotics include but are not limited to metronidazole, amoxycillin, tetracycline, erythromycin, clarithromycin or tinidazole.

In some examples, the vaccine of any preceding aspect further comprises a pharmaceutically acceptable carrier (such as, for example, including but not limited to nanoparticle or liposome), an adjuvant (such as, for example, including but not limited to alum, aluminum hydroxide, aluminum phosphate, potassium aluminum sulfate, calcium phosphate hydroxide, Freund's complete adjuvant, MONTANIDE™, Freund's incomplete adjuvant, iscoms, iscom matrix, ISCOMATRIX™ adjuvant, MATRIX M™ adjuvant, MATRIX C™ adjuvant, MATRIX Q™ adjuvant, AbISCO™-100 adjuvant, AbISCO™-300 adjuvant, ISCOPREP™, an ISCOPREP™ derivative, adjuvant containing ISCOPREP™ or an ISCOPREP™ derivative, QS-21, a QS-21 derivative, and an adjuvant containing QS-21 or a QS21 derivative) or a combination thereof.

In some examples, disclosed herein is a vector comprising a polynucleotide sequence encoding a Clostridioides difficile (C. difficile) Cwp2 protein or a fragment thereof. The polynucleotide sequence encoding the Cwp2 protein may be obtained from one or more C. difficile strains, including but not limited to RT027, RT078, RT017, RT012, RT003, or RT009. As disclosed herein, the polynucleotide sequence may comprise a sequence as set forth in SEQ ID NO: 1 or a sequence exhibiting at least 95%, 98%, 99%, or 99.8% identity thereto.

A “vector” refers to any vehicle that carries a polynucleotide into a cell for the expression of the polynucleotide in the cell. The vector may be, for example, a plasmid, a virus, a phage particle, or a nanoparticle. Once transformed into a suitable host, the vector may replicate and function independently of the host genome, or may in some instances, integrate into the genome itself. In some examples, the vector is a DNA construct containing a DNA sequence which is operably linked to a suitable control sequence capable of effecting the expression of the DNA in a suitable host cell. Such control sequences can include a promoter to effect transcription, an optional operator sequence to control such transcription, a sequence encoding suitable mRNA ribosome binding sites, and sequences which control the termination of transcription and translation. In some examples, the vector is a lipid nanoparticle. Lipid nanoparticles can be used to deliver mRNA to a host cell for expression of the mRNA in the host cell.

In some examples, the expression vector comprises a plasmid or a virus or viral vector. A plasmid or a viral vector can be capable of extrachromosomal replication or, optionally, can integrate into the host genome. As used herein, the term “integrated” used in reference to an expression vector (e.g., a plasmid or viral vector) means the expression vector, or a portion thereof, is incorporated (physically inserted or ligated) into the chromosomal DNA of a host cell. As used herein, a “viral vector” refers to a virus-like particle containing genetic material which can be introduced into a eukaryotic cell without causing substantial pathogenic effects to the eukaryotic cell. A wide range of viruses or viral vectors can be used for transduction but should be compatible with the cell type the virus or viral vector are transduced into (e.g., low toxicity, capability to enter cells). Suitable viruses and viral vectors include adenovirus, lentivirus, retrovirus, among others. In some examples, the expression vector encoding a chimeric polypeptide is a naked DNA or is comprised in a nanoparticle (e.g., liposomal vesicle, porous silicon nanoparticle, gold-DNA conjugate particle, polyethyleneimine polymer particle, cationic peptides, etc.).

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol. 4:1528-1533 (1984); Varga et al., J. Virology 65:6061-6070 (1991); Wickham et al., Cell 73:309-319 (1993)).

Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, CA, which can contain the herpes simplex virus thymidine kinase gene, HSV-tk, and/or a marker gene, such as the gene encoding the green fluorescent protein, GFP.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus.

Typically, the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorporated by reference for material related to the AAV vector.

In some examples, the vector disclosed herein may be a plasmid or an expression vector. As disclosed herein, the vector may be formulated with a pharmaceutically acceptable carrier, which may include nanoparticles or liposomes, thereby enhancing delivery and stability. Also disclosed herein, the vector may be incorporated into a viral vector system, such as an adenovirus vector, to facilitate gene delivery.

In some examples, disclosed herein is an immunogenic composition comprising a C. difficile Cwp2 protein or a fragment thereof, in combination with a pharmaceutically acceptable carrier, an adjuvant, or a combination thereof. In some examples, the adjuvant may be selected from aluminum hydroxide, aluminum phosphate, potassium aluminum sulfate, calcium phosphate hydroxide, Freund's complete adjuvant, MONTANIDE™, Freund's incomplete adjuvant, ISCOMS™, ISCOMATRIX™ adjuvant, MATRIX M™ adjuvant, MATRIX C™ adjuvant, MATRIX Q™ adjuvant, AbISCO™-100 adjuvant, AbISCO™-300 adjuvant, ISCOPREP™, QS-21, or derivatives thereof.

As disclosed herein, the immunogenic composition may be derived from C. difficile strains including RT027, RT078, RT017, RT012, RT003, or RT009. The Cwp2 protein included in the composition may have a sequence as set forth in SEQ ID NO: 18 or exhibit at least 95%, 98%, 99%, or 99.8% identity to the reference sequence. The immunogenic composition is capable of eliciting at least a B cell response, a CD4+ T cell response—including Th1, Th2, or Th17 responses—or a CD8+ T cell response. Furthermore, the pharmaceutically acceptable carrier may comprise nanoparticles or liposomes to improve immunogenicity.

In some examples, disclosed herein is a vaccine for preventing C. difficile infection, comprising an isolated Cwp2 protein, a vector as disclosed herein, or an immunogenic composition. In some examples, the vaccine induces an adaptive immune response against all strains of C. difficile. As disclosed herein, the vaccine may also be combined with at least one other pharmaceutical product, such as an antibiotic, which may include metronidazole, amoxicillin, tetracycline, erythromycin, clarithromycin, or tinidazole.

In agreement with serotyping studies [Delmee et al., 1986, 1990], which indicated that the production of an effective vaccine based on the slpA product is feasible. In some examples, disclosed herein, is an immunogenic composition or a vaccine comprising all variant of slpA genes and their products are included, individually and combined, fragments, mutants and derivatives.

In some examples, the immunogenic composition or the provides the combination of immunodominant epitopes from the slpA gene products from various serotypes into a single vaccine. In this way a single vaccine may be used to immunize against several different C. difficile strains. A vaccine elicits an intense antibody response against many infecting types would be therapeutically very valuable.

A recombinant protein, or isolated protein, encoding contiguous immunodominant epitopes from C. difficile surface components may be made for use in the vaccine. The recombinant protein may serve as the active component in a vaccine, or recombinant DNA encoding the recombinant protein may be inserted into an appropriate expression system for the generation of a recombinant peptide vaccine in a suitable host.

Recombinant DNA encoding the recombinant protein can be generated by PCR amplification of the DNA encoding peptide regions of interest, incorporating cleavage sites for restriction endonucleases into the primers. The amplified fragments can thus be cleaved to generate compatible ends, and spliced together to create a recombinant DNA.

The dominant epitopes may be identified by cleavage of the slpA products into fragments by agents which cleave at known sites, and by immunoblotting with homologous patient serum. Immunodominant peptides may be tested for their capacity to stimulate T-cell proliferative responses in vitro, using mouse splenic T-cells.

### Methods

In some examples, disclosed herein is a method of treating, inhibiting, reducing, decreasing, ameliorating, and/or preventing C. difficile infection in a subject, comprising administering to the subject a therapeutically effective dose of a vaccine, wherein the vaccine comprises an isolated protein or a vector (such as, for example, including but not limited to a plasmid, an expression vector or a viral vector), wherein the isolated protein or the vector comprises a C.difficile Cwp2 protein or a fragment thereof.

In some examples, disclosed herein is a method of treating a subject with a C. difficile infection, comprising administering to the subject a therapeutically effective dose of an isolated Cwp2 protein, a vector, an immunogenic composition, or a vaccine as disclosed herein. In some examples, the subject may receive at least one therapeutically effective dose. Additionally, the method may involve the co-administration of another pharmaceutical product, such as an antibiotic, selected from metronidazole, amoxicillin, tetracycline, erythromycin, clarithromycin, or tinidazole. The therapeutically effective dose may be administered via intravenous, intramuscular, intraperitoneal, intradermal, or subcutaneous routes.

In some examples, disclosed herein is a method of immunizing a subject against C. difficile infection by administering a therapeutically effective dose of the vaccine as disclosed herein. In some examples, at least one dose of the vaccine is administered, and the vaccine may also comprise another pharmaceutical product such as an antibiotic. The vaccine may be administered intravenously, intramuscularly, intraperitoneally, intradermally, or subcutaneously.

In some examples, the vaccine is a nucleotide based vaccine (DNA and mRNA). DNA vaccination involves immunization with recombinant DNA encoding the antigen or epitope of interest, cloned in a vector which promotes high level expression in mammalian cells. Typically, the vector is a plasmid vector which is also replicated in a prokaryotic vector such as Escherichia coli, so that the DNA can be produced in quantity. Following immunization, the plasmid enters a host cell, where it remains in the nucleus, and directs synthesis of the recombinant polypeptide. The polypeptide stimulates the production of neutralizing antibodies, as well as activating cytotoxic T-cells.

In some examples, an effective amount of an immunogenic composition or vaccine comprising a protein contains about 0.05 to about 1500 μg protein, about 10 to about 1000 μg protein, about 30 to about 500 μg, or about 40 to about 300 pg protein, or any integer between those values. For example, a protein may be administered to a subject at a dose of about 0.1 μg to about 200 mg, e.g., from about 0.1 μg to about 5 μg, from about 5 μg to about 10 μg, from about 10 μg to about 25 μg, from about 25 μg to about 50 μg, from about 50 μg to about 100 μg, from about 100 μg to about 500 μg, from about 500 μg to about 1 mg, or from about 1 mg to about 2 mg, with optional boosters given at, for example, 1 week, 2 weeks, 3 weeks, 4 weeks, two months, three months, 6 months and/or a year later.

In some examples, an effective amount of an isolated protein or immunogenic composition for passive or active immunization ranges from about 0.001 to about 30 mg/kg body weight, for example, about 0.01 to about 25 mg/kg body weight, about 0.1 to about 20 mg/kg body weight, about 1 to about 10 mg/kg, or about 10 mg/kg to about 20 mg/kg.

An isolated protein, immunogenic composition, vaccine, or vector may also be administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid). For prophylactic purposes, the amount of peptide in each dose is selected as an amount which induces an immunoprotective response without significant adverse side effects in a typical vaccine. Following an initial vaccination, subjects may receive one or several booster immunizations adequately spaced.

In some examples, disclosed herein is a method of producing a C. difficile homologous immunogen vaccine, comprising separating a broad group of C. difficile strains into homology groups based on similarities in toxinotypes, ribotypes, or sequence types for their Cwp2 protein; identifying at least a portion of the Cwp2 protein sequence for each homology group that has a sequence identity in excess of 60% to all other members of the homology group; and preparing a vaccine including at least a portion of the Cwp2 protein sequence from at least one homology group. As disclosed herein, the vaccine may elicit at least a B cell response, a CD4+ T cell response—including Th1, Th2, or Th17 responses—or a CD8+ T cell response.

Also disclosed herein, is a method of inhibiting C. difficile colonization in a subject, comprising administering to the subject a therapeutically effective dose of a vaccine comprising an isolated protein or a vector, wherein the isolated protein or vector comprises a C. difficile Cwp2 protein or a fragment thereof. In some examples, the vaccine further comprises a pharmaceutically acceptable carrier, an adjuvant, or a combination thereof. The adjuvant may be alum, and the pharmaceutically acceptable carrier may comprise a nanoparticle or a liposome.

In some examples, C. difficile strains may include RT027, RT078, RT017, RT012, RT003, or RT009. The C. difficile Cwp2 protein may have a sequence as set forth in SEQ ID NO: 18 or a sequence with at least 95%, 98%, 99%, or 99.8% identity thereto. The vector may comprise a polynucleotide encoding the Cwp2 protein or a fragment thereof, wherein the polynucleotide comprises a sequence as set forth in SEQ ID NO: 1 or a sequence having at least 95%, 98%, 99%, or 99.8% identity thereto. In some examples, the vector may be a plasmid or an expression vector, and it may comprise a viral vector such as an adenovirus vector.

In some examples, disclosed herein is a method of reducing recurrence of C. difficile infection in a subject by administering a therapeutically effective dose of a vaccine, wherein the vaccine comprises an isolated protein or a vector comprising a C. difficile Cwp2 protein or a fragment thereof. In some examples, the vaccine further comprises a pharmaceutically acceptable carrier, an adjuvant, or a combination thereof. The adjuvant may be alum, and the pharmaceutically acceptable carrier may comprise a nanoparticle or a liposome.

### EXAMPLES

The following examples are set forth below to illustrate the compounds, systems, methods, and results according to the disclosed subject matter. These examples are not intended to be inclusive of all examples of the subject matter disclosed herein, but rather to illustrate representative methods and results. These examples are not intended to exclude equivalents and variations of the present invention which are apparent to one skilled in the art.

### Example 1: Domain Architecture of Cell Wall Protein (Cwp2)

Clostridioides difficile is a Gram-positive, spore-forming enteric pathogen that causes a range of intestinal disorders and is a significant public health concern worldwide. Symptoms of C. difficile infection (CDI) can vary from diarrhea and intestinal inflammation to pseudomembranous colitis and even death. The commonly used treatments for CDI, which include a limited number of antibiotics such as fidaxomicin, vancomycin, and metronidazole, are not fully effective and are often associated with high recurrence rates (15-35%). Active vaccination offers a promising approach to preventing CDI and its recurrence; however, no vaccine against CDI has been licensed to date.

The symptoms of CDI are primarily caused by two major C. difficile toxins, toxin A (TcdA) and toxin B (TcdB), while binary toxin (CDT) produced by about 20% of C. difficile strains may also play a minor role in the pathogenesis of C. difficile. Consequently, significant efforts have been made to develop vaccines targeting TcdA/TcdB through parenteral immunizations, including two vaccine candidates in clinical trials: VLA84 and the genetically modified TcdA and TcdB from Pfizer. However, given that C. difficile is an enteric pathogen with a high rate of infection recurrence, effective vaccines should not only target the toxins but should also block C. difficile colonization. To address this, the focus was on identifying novel C. difficile surface colonization and adhesion factors that could be investigated as potential components of an effective vaccine against CDI and its recurrence.

The surface layer (S-layer) of C. difficile contains over 30 proteins, with the majority of the S-layer formed by SlpA, which is comprised of low and high molecular weight S-layer proteins (LMW SLP and HMW SLP). The cwp2 gene is found within the slpA locus, part of a cluster of S-layer-associated genes surrounding slpA. Cell wall protein 2 (Cwp2) contains a functional region (Cwp2_A) that includes domains 1, 2, and 3, as well as three cell wall binding domains (CWB1, CWB2, and CWB3) (FIG. 1), a conserved feature for C. difficile S-layer proteins. Cwp2 is recognized by nearly all CDI patient sera, indicating it is highly immunogenic. Additionally, a cwp2 knockout mutant in C. difficile strain 630 leads to increased TcdA release and impaired cellular adherence in vitro. Cwp2 is highly and constitutively expressed during normal C. difficile growth and is also found in the C. difficile spore coat. This study is aimed to analyze the homology and immunogenicity of Cwp2 and its protection efficacy as a vaccine candidate against CDI in mice.

### Example 2: Methods and Materials

### Animals

All studies followed the Guide for the Care and Use of Laboratory Animals of the National Institutes of Health and were approved by the Institutes Animal Care and Use Committee (IACUC). Wild-type C57BL/6 mice were used for the experiments.

### Phylogeny and Homology Analysis of Cwp2

C. difficile strains were sourced public databases and previous studies to create a dataset of strains from different ribotypes. The genomes of each strain were obtained from either GenBank (National Center for Biotechnology Information) or the Clostridioides difficile genome database from Enterobase. After mining the amino acid sequences for Cwp2 from each genome, MUSCLE alignments were performed of the sequences using MegaX set to default settings. Following sequence alignment, maximum likelihood phylogenetic trees with 100 bootstrap replicates were constructed, using MegaX. The cluster patterns from these phylogenetic trees guided the selection of specific Cwp2 sequences for domain analysis. The selected Cwp2 sequences were then aligned once more using the MUSCLE algorithm through the MPI Bioinformatics Toolkit server, and then the output files were visualized in Jalview.

### Expression and Purification of Recombinant Protein Cwp2_A

The functional domain Cwp2_A (amino acids 29-318) of the cwp2 gene from Clostridioides difficile R20291 was PCR-amplified and cloned into the NheI and XhoI sites of the expression vector pET28a in E. coli DH5a. The forward primer sequence is 5′-AGATGCTAGCCAGGTAAAAAAAGAAACAATAAC-3′ (SEQ ID NO: 3), and the reverse primer is 5′-GGTGCTCGAGTTATTCTAATGCAGCTTTGGCAT-3′ (SEQ ID NO: 4). The Cwp2_A protein fragment, containing an N-terminal His-tag, was purified using Ni-affinity chromatography. Briefly, the BL21 culture pellet (from 1000 ml culture) was resuspended in 40 ml lysis buffer (300 mM NaCl, 20 mM imidazole, 20 mM NaH₂PO₄, 500 μM EDTA, protease inhibitor cocktail; Cat #P8849, Sigma) adjusted to pH 8.0. Cells were disrupted via sonication, and the lysate was centrifuged at 15,000×g for 20 minutes. The resulting supernatant was passed through a nickel-charged HiTrap chelating HP column (Amersham Biosciences, Piscataway, NJ). The bound His-tagged Cwp2_A protein was eluted using a buffer containing 250 mM imidazole, 300 mM NaCl, and 20 mM NaH₂PO₄, pH 8.0. The eluted protein was subsequently desalted and further purified using a HiTrap Q column (Amersham Biosciences), where Cwp2_A was eluted via a NaCl gradient. Protein purity was confirmed by SDS-PAGE analysis, and the purified Cwp2_A protein was stored at −80° C. until use.

### Preparation of C. difficile Spores

Sporulation of the C. difficile R20291 strains were induced in Clospore medium. Briefly, an overnight 20 ml of C. difficile cultured in Columbia Broth was inoculated into 500 ml of Clospore medium, and incubated for 1-2 weeks at 37° C. in an anaerobic incubator. The spore suspension was centrifuged at 10,000 g for 20 min, and the pellet was washed 5 times with sterile water and suspended in 10 ml of ddH2O. The spore suspension was layered onto the top of 10 ml of 50% (wt/vol) sucrose in water in a 15-ml tube. The gradient was centrifuged at 3200×g for 20 min, after which the spore pellet at the bottom was washed five times to remove the sucrose and was resuspended in water. All spore preparations were >99% pure, free of vegetative cells and debris. The spore concentration was determined by serial dilution on TCCA or BHI plates.

### Mouse Immunization and Mouse Model of CDI

The Cwp2_A protein was treated with endotoxin removal resin according to the manufacturer's instructions (Cat #P188274, Thermo Scientific) prior to immunization. For animal immunization experiments, Cwp2_A (10 or 20 μg) was administered in PBS with aluminum (Imject Alum, cat #77161, Thermo Scientific) as an adjuvant. Thoroughly mixed Imject Alum was drop wisely added to Cwp2_A solution while mixing so the final volume ratio of Imject Alum to Cwp2_A is 1:2 (e.g., 100 μl of Imject Alum were added to 200 μl of immunogen); the mixture was continuously mixed for 30 minutes after adding the Imject Alum.

Mice (n=10, with 5 males and 5 females, 6 weeks old) were immunized three times at 12-day intervals via intraperitoneal (i.p.) injection. Control mice received equivalent volumes of PBS. Sera and fecal samples were collected. Immunization experiments were repeated once. Seven days after the third immunization, both immunized and control mice were provided with drinking water containing a mixture of five antibiotics: kanamycin (40 mg/kg), gentamycin (3.5 mg/kg), colistin (4.2 mg/kg), metronidazole (21.5 mg/kg), and vancomycin (4.5 mg/kg) for four days. This was followed by two days of autoclaved water. Mice then received a single intraperitoneal injection of clindamycin (10 mg/kg) prior to being challenged with 10⁶ C. difficile R20291 spores per mouse via oral gavage. Post-infection mice were monitored daily for one week to assess survival, weight changes, diarrhea, and other disease symptoms. Diarrhea was defined by the presence of wet tails and loose or watery feces. Mouse mortality was recorded as the number of mice that died post-infection or were euthanized due to weight loss exceeding 20%.

### ELISA for Anti-Cwp2_A IgG/A

Briefly, Costar 96-well ELISA plates were coated with 100 μl/well of Cwp2_A (0.5 μg/ml) and incubated overnight at 4° C. Unbound material was washed off, and the wells were blocked with 300 μl of blocking buffer (PBS+5% dry milk) for 2 hours at room temperature. Subsequently, 100 μl of 10-fold diluted sera or fecal samples were added to each well and incubated for 1.5 hours at room temperature. After washing with PBS, 100 μl of either HRP-conjugated mouse IgG (1:3000) or IgA (1:3000) was added and incubated for 30 minutes to 1 hour. Following another PBS wash, TMB substrate was added to induce color development for 5-30 minutes at room temperature. The reaction was terminated by adding H₂SO₄, and the optical density (OD) at 450 nm was measured using a spectrophotometer. The anti-Cwp2_A IgG/A titer for each sample was defined as the dilution factor at which the OD₄₅₀nm was at least 2-fold higher than that of serum samples from non-immunized mice.

### Quantification of C. difficile Spores in Mouse Feces

Fecal samples were collected on days 0, 1, 3, 5, and 7 post-infection. Fifty milligrams of feces were suspended in 500 μl of sterile water and incubated at 4° C. for 16 hours. To eliminate vegetative cells, the samples were treated with 500 μl of absolute ethanol (Sigma Aldrich) for 1 hour at room temperature. After vortexing, the samples were serially diluted and plated onto BHI medium supplemented with taurocholate (0.1% w/v), cefoxitin (8 μg/ml), and D-cycloserine (250 μg/ml). Plates were incubated anaerobically at 37° C. for 48 hours, and colonies were subsequently counted. Results were expressed as CFU per gram of feces.

### ELISA for TcdA and TcdB

Following the challenge with Clostridioides difficile spores, fecal samples were collected and dissolved in PBS (0.1 g/ml) containing a protease inhibitor cocktail. The samples were centrifuged, and the resulting supernatants were collected to determine TcdA and TcdB concentrations using ELISA. Briefly, 96-well Costar microplates were coated overnight at 4° C. with 100 μl of anti-TcdA and anti-TcdB antibodies (1 μg/ml each) prepared in phosphate-buffered saline (PBS). The following day, wells were blocked with 300 μl of blocking buffer (PBS+5% dry milk) for 2 hours at room temperature. Standards and samples (100 μl each) were added in duplicate and incubated for 90 minutes at 25° C. After washing, HRP-conjugated chicken anti-C. difficile TcdA/TcdB antibodies (1:5,000 dilution in PBS; Gallus Immunotech, Shirley, MA) were applied for 30 minutes at room temperature. Following a final wash, TMB microwell peroxidase substrate was added and incubated for 20 minutes at room temperature in the dark. The reaction was stopped by adding 2 N H₂SO₄, and absorbance was measured at 450 nm using a plate reader.

### Adherence Inhibition Assays

The adherence of Clostridioides difficile R20291 vegetative cells to human gut epithelial cells was evaluated as described previously described [38,43]. Briefly, HCT-8 cells were cultured to 95% confluence (1×10\hat{ }5 cells/well) in a 24-well plate and transferred to an anaerobic chamber. The cells were infected with 1.5×10\hat{ }6 log-phase R20291 vegetative cells at a multiplicity of infection (MOI) of 15:1, followed by incubation at 37° C. for 100 minutes. Prior to infection, R20291 vegetative cells were preincubated with anti-Cwp2_A sera at dilutions of 1/50, 1/100, and 1/200 for 30 minutes. Following incubation, the cell-R20291 mixture was washed three times with 1× PBS by centrifugation at 800×g for 1 minute to remove non-adherent cells. Supernatants from each wash step were collected, and non-adherent R20291 cells were enumerated on pre-reduced BHI agar. Control experiments included R20291 cells incubated with PBS or preimmune sera ( 1/25 dilution). Adhesion assays were performed in triplicate. The percentage of R20291 adherence was calculated using the formula: Adherence (%)=100×(initial CFU/ml−eluted CFU/ml)/initial CFU/ml.

### Determination of Cytokine Expression by Real-Time PCR

Mice spleen (n=5) were obtained from CWP2-immunized and control (non-immunized) mice 13 days after the second immunization. Splenocytes were isolated through 70 μm and 40 μm cell strainer using syringe plunges to prepare single cell suspensions. Cells were counted and seeded in RPMI 1640 medium (Thermofisher, USA) containing 10% FBS and incubated at 37° C. and 5% CO2. The cells were then pulsed with 10 μg/ml Cwp2 for 72 hours and used for flow cytometry analysis below. A portion of immunogen-pulsed cells were collected for expression analysis of IL-4, IL-5, IL-17, IFN-γ and TNF-α by real-time PCR. Primers used (Table 1) are from published papers. The GAPDH gene was chosen as the reference for internal standardization. GAPDH is a well-accepted housekeeping gene, which has been used in other studies.

Real-time PCR was performed on CFX Opus 96 real-time PCR system (Bio Rad, USA). SYBR Green/ROX qPCR Master mix (Thermo Fisher) was used according to the manufacturer's protocol. Reaction conditions were as follows: 95 C for 10 sec, followed by 40 cycles of 95 C for 15 s, 60 C for 30 sec. Reaction of each sample was performed in triplicate. Dissociation analysis was performed at the end of each PCR reaction to confirm the amplification specificity. After the PCR program, data were analyzed and quantified with the comparative Ct method (2-ΔΔCt) based on Ct values for cytokine genes and GAPDH in order to calculate the relative mRNA expression level.

TABLE 1
Primers used for real-time PCR

| | Forward primer | Reverse primer |
|---|---|---|
| Target | (5′ to 3′) | (5′ to 3′) |
| GAPDH | TGCACCACCAACTGCTTAG | GGATGCAGGGATGATGTTC |
| | (SEQ ID NO: 5) | (SEQ ID NO: 6) |
| IFN-γ | AAAGAGATAATCTGGCTCTGC | GCTCTGAGACAATGAACGCT |
| | (SEQ ID NO: 7) | (SEQ ID NO: 8) |
| TNF-α | CCACCACGCTCTTCTGTCTAC | AGGGTCTGGGCCATAGAACT |
| | (SEQ ID NO: 9) | (SEQ ID NO: 10) |
| IL-17 | GGAGAAAGCGGATACCAA | TGTGAGGACTACCGAGCC |
| | (SEQ ID NO: 11) | (SEQ ID NO: 12) |
| IL-4 | TCTCGAATGTACCAGGAGCCA | AGCACCTTGGAAGCCCTACA |
| | TATC (SEQ ID NO: 13) | GA (SEQ ID NO: 14) |
| IL-5 | AGCACAGTGGTGAAAGAGACC | TCCAATGCATAGCTGGTGAT |
| | TT (SEQ ID NO: 15) | TT (SEQ ID NO: 16) |

### Antigen Specific T Cells Proliferation Assays

The spleen cells collect above were pulsed with 10 μg/ml CWP2 for 72 hours. The cells were stained by antibodies against CD3, CD4 and CD8 (BioLegend) by flow cytometry analysis.

### Statistical Analysis

Animal survival was assessed using Kaplan-Meier survival analysis, with statistical significance determined using the log-rank test. For comparisons between two groups, the non-parametric Student's t-test was employed, while one-way analysis of variance (ANOVA) with post-hoc Bonferroni tests was used for comparisons involving more than two groups.

Results are presented as means±standard error of the mean (SEM), and differences were considered statistically significant at p<0.05(*). All statistical analyses were conducted using GraphPad Prism software. Non-parametric tests were chosen because the data did not follow a normal distribution.

### Example 3: The Functional Domain of Cwp2 is Highly Immunogenic Based on B Cell Epitope Analysis

To assess immunogenicity, a B cell epitope analysis was conducted using the BepiPred-2.0 server. The functional domain of Cwp2 (aa 27-295, Cwp2_A), which includes domains 1, 2, and 3, contains the majority of immunogenic peptides (highlighted in yellow). In contrast, the cell wall binding regions (CWB1, CWB2, and CWB3) have very few low-immunogenic peptides. This indicates that Cwp2_A is potentially highly immunogenic (FIG. 2).

### Example 4: Phylogeny and Homology of Cwp2 across C. difficile Strains from Various Toxinotypes and Ribotypes

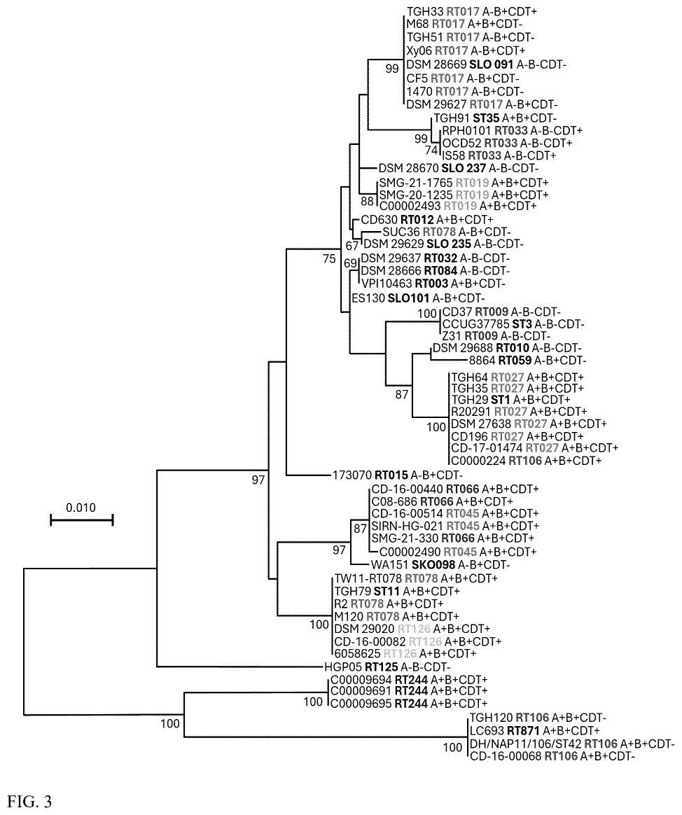

To identify effective vaccine candidates against CDI, immunogens should be conserved across different C. difficile strains. To this end, the phylogeny and homology of the Cwp2 protein was investigated in major toxinotypes as well as ribotypes (R.T.) and sequence types (S.T.). Maximum likelihood phylogenetic trees were generated using Cwp2 amino acid sequences from various C. difficile strains to determine any correlation between sequence similarity and either the source strain's R.T. or toxinotype (FIG. 3). Our analysis revealed a strong correlation between C. difficile R.T. and Cwp2 relatedness. Most strains within a given R.T. have identical Cwp2 sequences (e.g., RT017, RT033, RT027). Only three ribotypes (RT106, RT045, RT078) do not have identical Cwp2 sequences, but the Cwp2 sequences within these ribotypes generally cluster close together (e.g., three out of four Cwp2 sequences for either RT016 or RT078 cluster together). For RT106 strains, strain C00000224 encodes a Cwp2 identical to those found in RT027 strains, while the other two RT106 Cwp2 sequences cluster in a different branch of the tree. In RT045 strains, C00002490 Cwp2 has an aspartic acid (D) instead of an asparagine (N) at position 214 compared to the other two RT045 Cwp2 sequences. Additionally, ribotype may indicate a lack of Cwp2, as all three RT023 strains examined (SIRN_ST-001, CD-16-00530, CD-15-00694) did not encode Cwp2, consistent with previous reports that Cwp2 is absent in some C. difficile strains. Unlike the correlation observed between Cwp2 and ribotype, C. difficile toxinotype does not show a strong correlation with Cwp2 phylogeny.

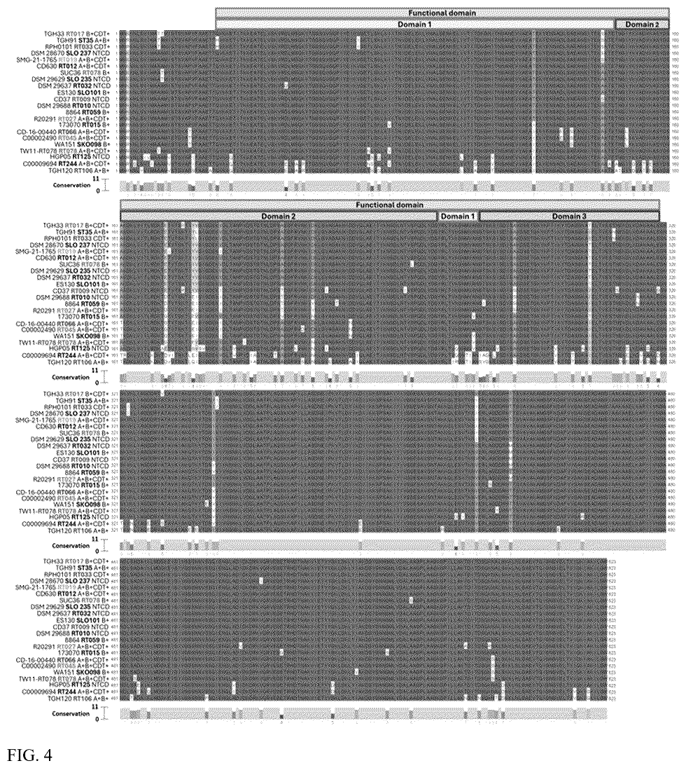

To more thoroughly examine Cwp2 sequence diversity, the sequences of the whole Cwp2 protein (FIG. 4) were aligned using MUSCLE and visualized the output in Jalview software. Representative sequences were selected from each cluster identified in the phylogenetic trees (FIG. 3), ensuring that all non-identical sequences were included in the analysis. Upon aligning and visualizing twenty-two selected Cwp2 sequences, it was found that they share 79% identical residues (130 out of 623) with a pairwise homology level ranging from 89% to 100% (FIG. 4). Among the non-identical residues, 59% of the variations were located in the functional region (aa 23-318) despite this region comprising only 46% of the protein. The pairwise homology level within the functional region ranges from 84% to 100%, while the remaining regions show a pairwise homology range of 93% to 100%. The functional region of Cwp2 is predicted to mediate the adhesive properties of the protein. Similarly, other Cwp proteins encoded by the SlpA locus, such as Cwp 6, Cwp 8, and LMW SLP, utilize their functional region for adhesion, and these surface proteins display significant inter-strain variability within their functional regions

Based on structural similarities with Cwp8, prior studies argue that domain 2 is surface exposed while domains 1 and 3 are located within the outer C. difficile membrane. Since Cwp8 domain 2 is known to be the most variable domain within the protein, the same is hypothesized for Cwp2. In both cases, this variability is thought to facilitate immune evasion. In the analysis, domain 2 has a greater total number of non identical residues between the strains analyzed (36) when compared to domain 1 (23) or domain 3 (18). However, the ratio of non-identical residues to domain length is lowest in domain 2 ( 36/399, or 9%) compared to domain 1 ( 23/128, 18%) or domain 3 ( 18/53, or 34%).

Cwp2 also possesses the trimeric cell wall-anchoring CWB domains (CWB1, CWB2, and CWB3), which are a shared feature of C. difficile S-layer proteins. Deletions of CWB2 prevent the anchoring of Cwp2 to the exterior of C. difficile, and Cwp2 also requires certain sequence patterns for correct anchoring as well. It was found that the trimeric CWB2 domains from Cwp6 and Cwp8 show moderate similarity (56 and 66% positives, respectively, see Table 1) with the C-terminal region of Cwp2 following the functional domain (amino acids 322-621). In FIG. 4, 13% (41/305) of residue variations between the strains are found in the approximate region of the CWB2 domains (following the functional region) despite this region comprising about 49% of the protein length.

In summary, Cwp2 is present in all toxinotypes and in most ribotypes and sequence types of all C. difficile strains, particularly the major clinically relevant ones (e.g., RT027, RT078, RT106, RT017). The Cwp2 amino acid sequences are generally identical within each ribotype, with a homology level of 89%-100% among all ribotypes and sequence types analyzed. The functional domain of Cwp2 is also highly conserved, though it exhibits more variability compared to the cell wall binding (CWB) regions.

### Example 5: Immunization of Mice with Cwp2 Functional Domain (Cwp2_A) Induces Significant Anti-Cwp2_A Antibody Responses in Mice and Provides Protection against C. difficile Infection

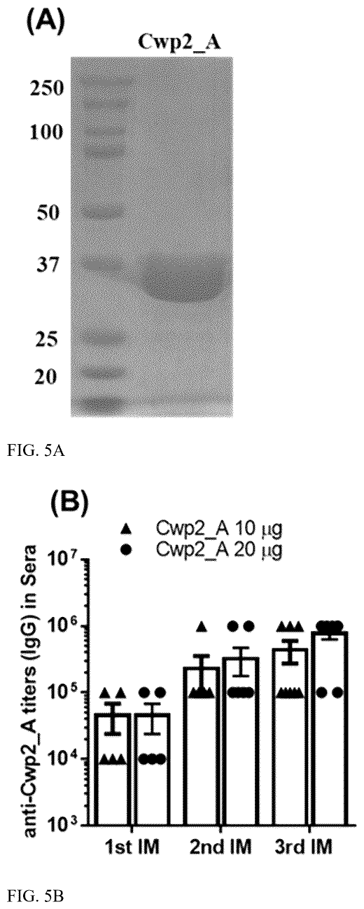

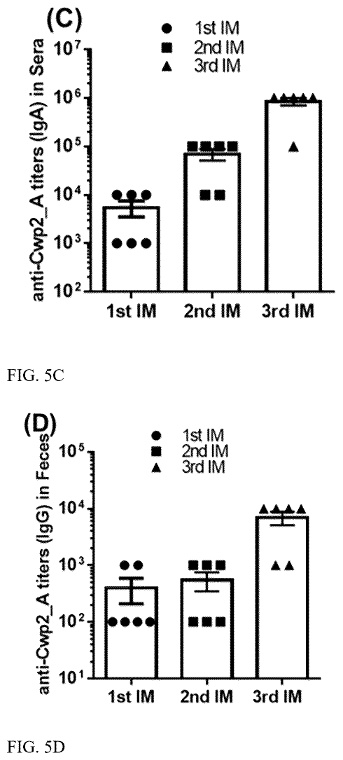

Given that the Cwp2_A region is predicted to be highly immunogenic and moderately conserved, Cwp2_A was chosen as a potential vaccine component. To explore this, recombinant Cwp2_A with a 6xHis tag was expressed in E. coli BL21 and purified to over 95% purity using Ni-affinity chromatography (FIG. 5A). Immunization of mice with 10 μg or 20 μg of Cwp2_A, combined with alum as an adjuvant and administered via the intraperitoneal route, induced high levels of IgG and IgA antibody responses against Cwp2_A in both sera and feces (FIGS. 5B, 5C, 5D, and 5E).

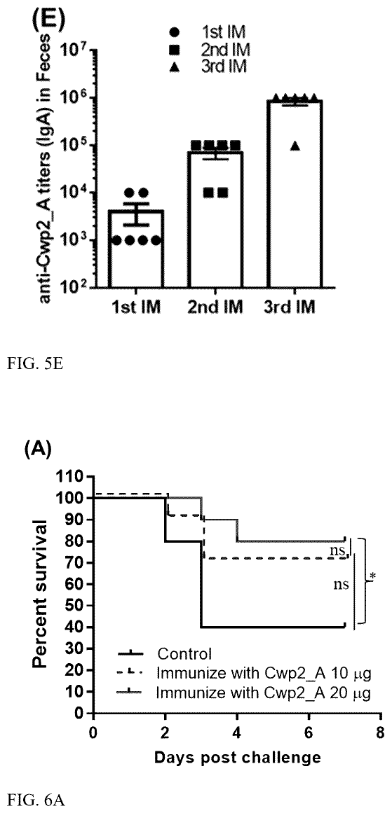

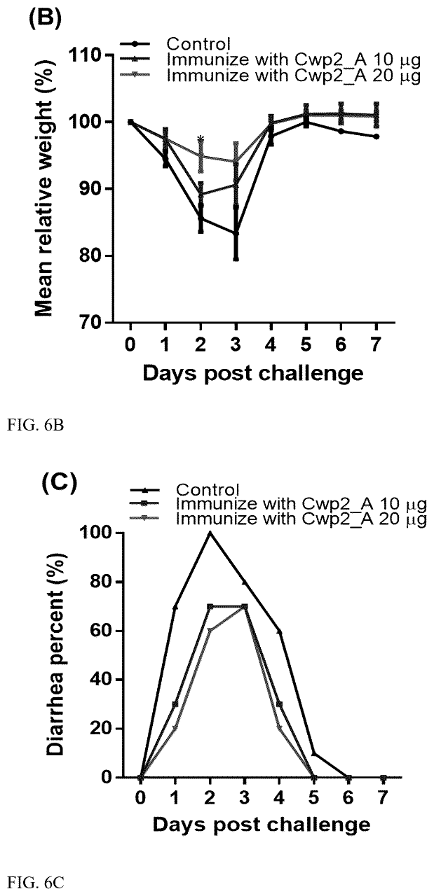

The protective efficacy of Cwp2_A immunization was further evaluated in a mouse model of CDI. Mice were immunized three times with either 10 μg or 20 μg of Cwp2_A at 12-day intervals and subsequently challenged with 10\hat{ }6 spores of C. difficile R20291, a hypervirulent ribotype 027 strain. In the vehicle (PBS)-immunized group, significant disease symptoms were observed in all mice, including weight loss (FIG. 6B) and severe diarrhea (FIG. 6C), with approximately 60% succumbing by day 4 (FIG. 6A). In contrast, Cwp2_A immunized mice exhibited milder disease symptoms, characterized by reduced weight loss (FIG. 6B) and lower rates of diarrhea (FIG. 6C), along with higher survival rates (70% for the 10 μg Cwp2_A group and 80% for the 20 μg Cwp2_A group; FIG. 6A).

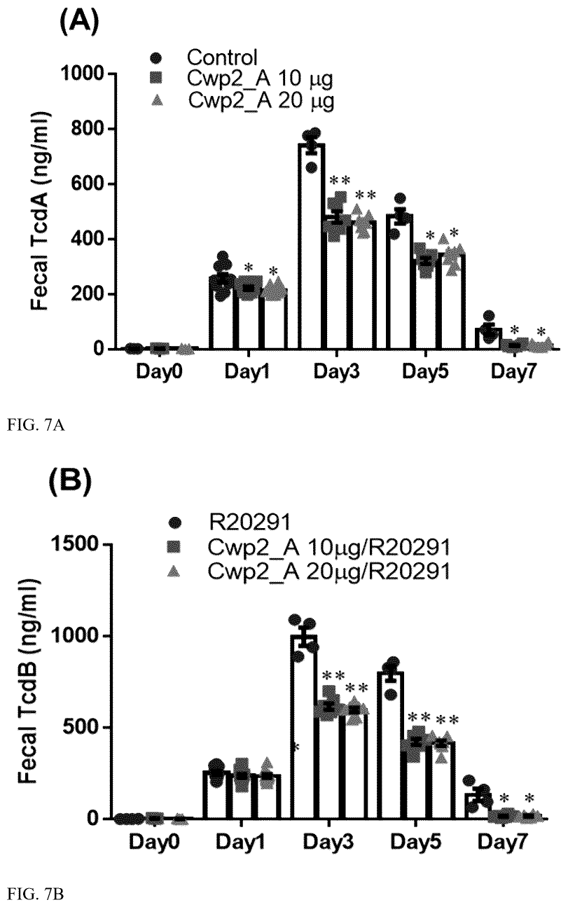

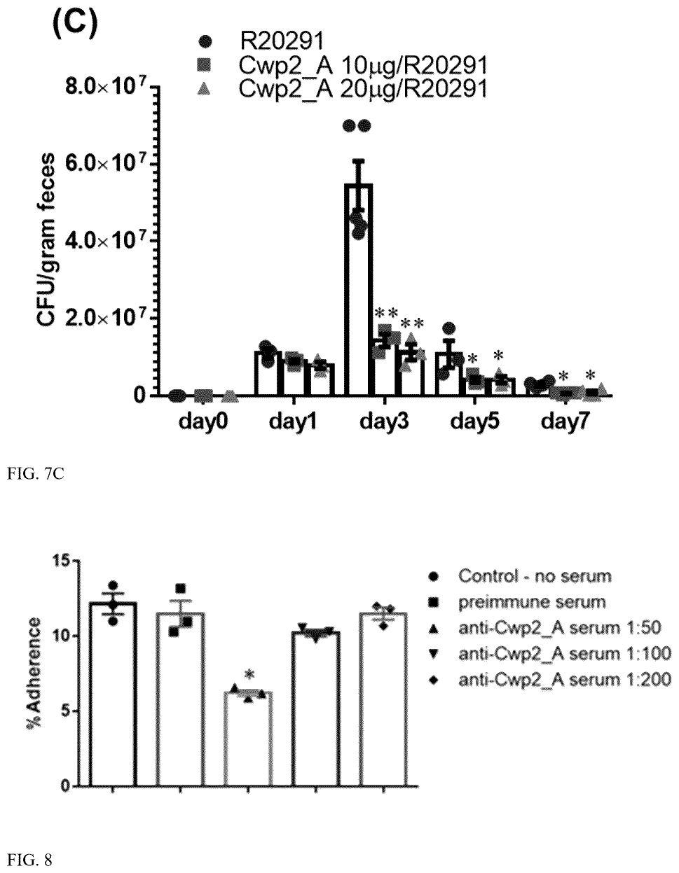

Cwp2_A-immunized mice excreted significantly lower amounts of toxin A (FIG. 7A) and toxin B (FIG. 7B) in their feces compared to the PBS-immunized group. Furthermore, fecal samples from Cwp2_A-immunized mice contained significantly fewer R20291 spores compared to those from the PBS-immunized group (FIG. 7C).

### Example 6: Anti-Cwp2_A Serum Inhibits the Binding of C. difficile to HCT8 Cells

Given that Cwp2_A is predicted to mediate Clostridioides difficile cell adhesion and that Cwp2_A immunization significantly reduced C. difficile spores in infected mice, anti-Cwp2_A antibodies may inhibit C. difficile adhesion to host cells. To test this, an in vitro adhesion assay was performed. When anti-Cwp2_A serum was diluted 1:50 in the cell medium, the adherence rate of C. difficile R20291 vegetative cells to HCT8 cells was significantly reduced (6.733±0.5239% vs. 12.17±0.6936%). At a 1:100 dilution, the adherence rate decreased to 10.22±0.223%, though this reduction was not statistically significant (FIG. 8).

### Example 7: Immunization of Mice with Cwp2_A Induces Th1- and Th17-Type Immune Responses

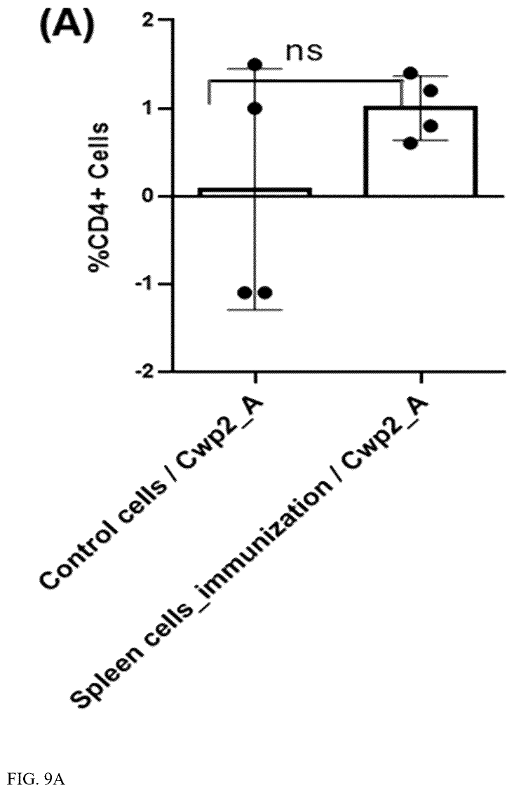

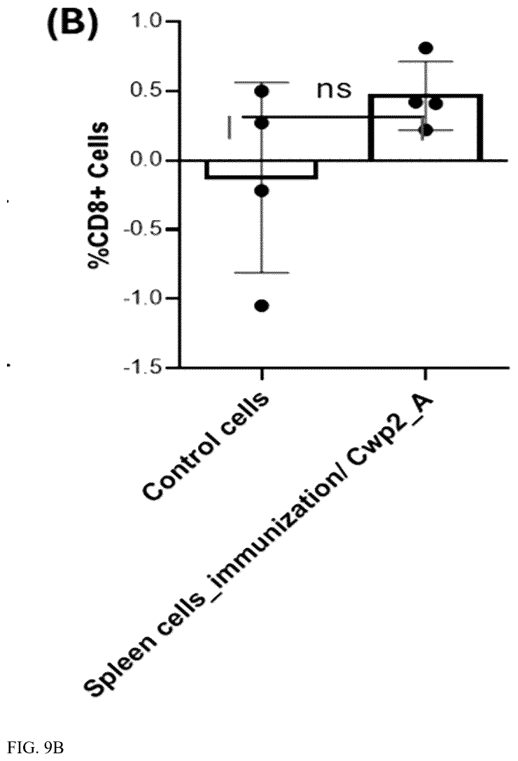

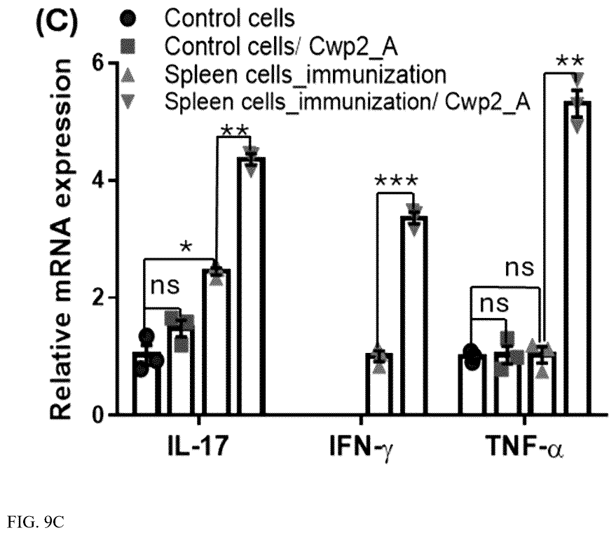

To evaluate the dominant subset of T cells in the spleen of immunized mice and assess whether the T cells could propagate after re-stimulation with corresponding antigens in vitro, splenocytes were pulsed with Cwp2_A at 10 μg/ml for 72 hours. Compared to the unimmunized control group, the percentage of both CD4+ and CD8+ T cells increased in immunized mice, though not statistically significant (FIGS. 9A and 9B). Additionally, CD4+ T cells propagated around 2 times more than CD8+ T cells, indicating a potentially dominant Th cell response in immunized mice in response to Cwp2_A. Since cytokines secreted by the activated T cells are indicators of the type of Th responses, the expression of IFN-γ and TNF-α (Th1 response), IL-17 (Th17 response), IL-4 and IL-5 (Th2 response) was assessed by qPCR in the splenocytes stimulated with Cwp2_A at 10 μg/ml for 6 hours. Expression of IL-17, IFN-γ and TNF-α in splenocytes was all significantly increased after immunization or re-stimulation (FIG. 9C). These data support Th1- and Th17-responses induced by Cwp2_A immunization.

### Discussion

The in-silico analysis showed that the functional domain (Cwp2_A) is highly immunogenic and is also conserved. The immunogenicity of Cwp2_A and its protective effect against CDI in mice was further evaluated. The data showed that the functional domain Cwp2_A is highly immunogenic and conserved. Immunization with Cwp2_A effectively protected mice against CDI and reduced C. difficile spore and toxin levels in the feces of mice challenged with C. difficile spores. Additionally, anti-Cwp2 serum inhibited the binding of C. difficile R20291 vegetative cells to HCT8 cells. The data suggest that Cwp2 is an important colonization factor for C. difficile both in vitro and in vivo and that it alone is a promising vaccine candidate against CDI.

C. difficile toxin-based vaccine candidates have failed in clinical trials. Considering that C. difficile is an enteric pathogen, causing high rates of CDI recurrence, effective vaccine candidates should target not only the toxins, but also the pathogen colonization to prevent primary and recurrent CDI and disease transmission. Furthermore, C. difficile vaccine candidates should induce both systematic and intestinal immune antibody responses. To this end, researchers have been actively exploring C. difficile surface components as potential vaccine components in combination with toxin-based vaccine candidates. So far, many surface components have been evaluated, which include surface proteins and colonization factors, such as SlpA, Cwp84, Cwp66, FliC, FliD, CdeC, CdeM, cell wall polysaccharides (PS-I, PS-II, PS-III), lipoprotein CD0873, chaperon protein DnaK, heat shock protein GroEL, BclA3, etc., all of which can induce protective immune responses against CDI in animals to different extents, but none of them have been evaluated in clinical trials. The study demonstrated that Cwp2, which is an abundant, rather conserved, and can be easily produced in large quantity, is highly immunogenic and induces significant protection against CDI as an immunogen, providing a promising option to target C. difficile colonization and transmission.

In addition, the current data showed that immunization of mice with Cwp2_A induced Th1 (IFN-γ and TNF-α), Th17 (IL-17) type immune responses, and Th2 (IL-4 & IL-5) response, as evaluated by qPCR based on splenocytes pulsed with antigen for 6 hours. Adjuvants used are alum, aluminum hydroxide (AH), liposomes and aluminum phosphate (AP). Further, highly conserved motifs and immune epitopes within the Cwp2_A sequence can enhance the effectiveness of vaccines, potentially in combination with those targeting C. difficile toxins and other colonization factors.

The current application demonstrates for the first time the potential of Cwp2_A as an effective vaccine candidate against CDI in mice, which will be a valuable contribution to the on-going combat against a serious infection caused by the multiple-antibiotic resistant C. difficile.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will appreciate that numerous changes and modifications can be made to the preferred examples of the invention and that such changes and modifications can be made without departing from the spirit of the invention. It is, therefore, intended that the appended claims cover all such equivalent variations as fall within the true spirit and scope of the invention.

SEQUENCES

SEQ ID NO: 1-Full cwp2 gene region (DNA)
atgaataaaaaaaatctttctgtaattatggctgctgcaatgataagtacatcagtagctccagtttttgctgcagaaactacacaggta
aaaaaagaaacaataactaagaaagaagctacagaactagtttcgaaagttagagatttaatgtctcaaaagtatactggtggttctc
aagttggacaaccaatatatgaaataaaagttggcgagactttatcaaaattaaaaataataactaatatagatgaattagagaaatta
gtaaatgctttgggagaaaataaagaacttattgtaactataacagataaagggcatataacaaatagtgcaaatgaagtagttgcag
aagcaactgaaaaatatgaaaattcagcagacctttccgctgaggctaattctataacagaaaaagctaaaactgaaactaatggaat
ttataaagttgcagatgtaaaagcttcatatgatagtgctaaagataagttagttataactttaagagataaaacagacacagtaacttct
aaaactatagagataggtattggtgatgaaaaaattgatttaacagcaaatccagttgattcaacgggaacaaacttagacccttctac
agaaggatttagagtaaataaaatcgttaaactaggtgtagcaggagctaaaaatattgatgatgtccaattagctgaaataactataa
aaaatagtgacctaaatacagtttcaccacaagatttatatgatggatatagattaactgttaaaggtaatatggtagcaaatggaacat
caaagtcaattagtgatatttcatcaaaagattcagaaacaggaaagtataaatttactattaagtatactgatgcatctggaaaagcaa
tagagcttactgtagaaagtactaatgaaaaagatttaaaagatgccaaagctgcattagaaggtaattcaaaggttaaattgata
gctggagatgatagatatgcaactgcagtggctatagcaaaacaaacaaaatatactgacaatatagttatagttaattca
aataaactagttgatggattagcagctacaccacttgctcaatctaaaaaagcacctatattattagcatccgataatgaaa
taccaaaagtaactttagattatataaaagatataattaagaaaagcccatcagctaaaatatatatagtaggtggagaat
cagcagtatcaaatacagctaaaaagcaattagaatcagtaactaagaatgttgaaagactagctggagatgatagacat
atgacttctgtagcagtagcaaaagctatggggtcttttaaagatgcatttgtagtaggtgcgaaaggggaggctgatgcta
tgagtatagctgccaaagctgctgaacttaaggctcctataatagtaaatggctggaatgatctttcagcagacgctatcaa
attgatggatggaaaagagattggtatagttggtggttctaacaatgtatctagtcaaattgaaaatcaacttgctgatgttg
ataaagatagaaaagttcaaagagttgaaggagaaacaagacacgatactaatgctaaggttatagaaacatattatgg
aaaattagataaactatatatagcaaaagatggatatggaaataatggtatgctagtagatgcattggcagcaggacctct
agcagcaggtaaaggtccaatacttctagctaaagctgatataacagactcacaaaggaatgcacttagtaaaaaattaa
atcttggtgcagaagtaactcaaataggtaatggagttgaattgacagtaatacaaaagatagctaaaatactaggttggt
aa
Signal peptide = underline
Transmembrane region = bold
SEQ ID NO: 2-Cwp2_A (DNA)
caggtaaaaaaagaaacaataactaagaaagaagctacagaactagtttcgaaagttagagatttaatgtctcaaaagtatactggtg
gttctcaagttggacaaccaatatatgaaataaaagttggcgagactttatcaaaattaaaaataataactaatatagatgaattagaga
aattagtaaatgctttgggagaaaataaagaacttattgtaactataacagataaagggcatataacaaatagtgcaaatgaagtagtt
gcagaagcaactgaaaaatatgaaaattcagcagacctttccgctgaggctaattctataacagaaaaagctaaaactgaaactaat
ggaatttataaagttgcagatgtaaaagcttcatatgatagtgctaaagataagttagttataactttaagagataaaacagacacagta
acttctaaaactatagagataggtattggtgatgaaaaaattgatttaacagcaaatccagttgattcaacgggaacaaacttagaccc
ttctacagaaggatttagagtaaataaaatcgttaaactaggtgtagcaggagctaaaaatattgatgatgtccaattagctgaaataa
ctataaaaaatagtgacctaaatacagtttcaccacaagatttatatgatggatatagattaactgttaaaggtaatatggtagcaaatg
gaacatcaaagtcaattagtgatatttcatcaaaagattcagaaacaggaaagtataaatttactattaagtatactgatgcatctggaa
aagcaatagagcttactgtagaaagtactaatgaaaaagatttaaaagatgccaaagctgcattagaa
SEQ ID NO: 3-Cwp2_A Forward primer
agatgctagccaggtaaaaaaagaaacaataac
SEQ ID NO: 4-Cwp2_A Reverse primer
ggtgctcgagttattctaatgcagctttggcat
SEQ ID NO: 5-GAPDH Forward Primer
tgcaccaccaactgcttag
SEQ ID NO: 6-GAPDH Reverse Primer
ggatgcagggatgatgttc
SEQ ID NO: 7- IFN-γ Forward Primer
aaagagataatctggctctgc
SEQ ID NO: 8-IFN-γ Reverse Primer
gctctgagacaatgaacgct
SEQ ID NO: 9- TNF-α Forward Primer
ccaccacgctcttctgtctac
SEQ ID NO: 10- TNF-α Reverse Primer
agggtctgggccatagaact
SEQ ID NO: 11-IL-17 Forward Primer
ggagaaagcggataccaa
SEQ ID NO: 12-IL-17 Reverse Primer
tgtgaggactaccgagcc
SEQ ID NO: 13-IL-4 Forward Primer
tctcgaatgtaccaggagccatatc
SEQ ID NO: 14-IL-4 Reverse Primer
agcaccttggaagccctacaga
SEQ ID NO: 15- IL-5 Forward Primer
agcacagtggtgaaagagacctt
SEQ ID NO: 16-IL-5 Reverse Primer
tccaatgcatagctggtgattt
SEQ ID NO: 17- Cwp2_A (AA)
QVKKETITKKEATELVSKVRDLMSQKYTGGSQVGQPIYEIKVGETLSKLKIITNIDE
LEKLVNALGENKELIVTITDKGHITNSANEVVAEATEKYENSADLSAEANSITEKA
KTETNGIYKVADVKASYDSAKDKLVITLRDKTDTVTSKTIEIGIGDEKIDLTANPVD
STGTNLDPSTEGFRVNKIVKLGVAGAKNIDDVQLAEITIKNSDLNTVSPQDLYDGY
RLTVKGNMVANGTSKSISDISSKDSETGKYKFTIKYTDASGKAIELTVESTNEKDLK
DAKAALE
SEQ ID NO: 18-Full cwp2 gene region (AA)
MNKKNLSVIMAAAMISTSVAPVFAAETTQVKKETITKKEATELVSKVRDLMSQKY
TGGSQVGQPIYEIKVGETLSKLKIITNIDELEKLVNALGENKELIVTITDKGHITNSA
NEVVAEATEKYENSADLSAEANSITEKAKTETNGIYKVADVKASYDSAKDKLVIT
LRDKTDTVTSKTIEIGIGDEKIDLTANPVDSTGTNLDPSTEGFRVNKIVKLGVAGAK
NIDDVQLAEITIKNSDLNTVSPQDLYDGYRLTVKGNMVANGTSKSISDISSKDSETG
KYKFTIKYTDASGKAIELTVESTNEKDLKDAKAALEGNSKVKLIAGDDRYATAVA
IAKQTKYTDNIVIVNSNKLVDGLAATPLAQSKKAPILLASDNEIPKVTLDYIKDIIKK
SPSAKIYIVGGESAVSNTAKKQLESVTKNVERLAGDDRHMTSVAVAKAMGSFKD
AFVVGAKGEADAMSIAAKAAELKAPIIVNGWNDLSADAIKLMDGKEIGIVGGSNN
VSSQIENQLADVDKDRKVQRVEGETRHDTNAKVIETYYGKLDKLYIAKDGYGNN
GMLVDALAAGPLAAGKGPILLAKADITDSQRNALSKKLNLGAEVTQIGNGVELTV
IQKIAKILGW

What is claimed is:

1. An isolated protein comprising a surface component of one or more strain of Clostridioides difficile (C.difficile), wherein the surface component comprises surface proteins, colonization factors, or combination thereof.

2. The isolated protein of claim 1, wherein the one or more strain of C.difficile is selected from a group consisting of a RT027 strain, a RT078 strain, a RT017 strain, a RT012strain, a RT003 strain, and a RT009 strain.

3. The isolated protein of claim 1, wherein the surface component is a surface layer protein comprising a cell wall protein 2 (Cwp2).

4. The isolated protein of claim 3, wherein the Cwp2 contains an amino acid sequence SEQ ID NO: 18, or a fragment thereof.

5. The isolated protein of claim 3, wherein the Cwp2 is encoded by a polynucleotide sequence SEQ ID NO: 1 or a sequence having at least 90% identity thereto.

6. The isolated protein of claim 3, wherein Cwp2 comprises a fragment Cwp2_A, wherein the Cwp2_A comprises a sequence from about amino acid number 29 to about amino acid number 318 of SEQ ID NO: 17 or a sequence having at least 95%, 98%, 99% or 99.8% identity thereto.

7. An immunogenic composition comprising:
a C. difficile Cwp2 protein or a fragment thereof; and
a pharmaceutically acceptable carrier, an adjuvant or a combination thereof.

8. The immunogenic composition of claim 7, wherein the adjuvant comprises alum, aluminum hydroxide or aluminum phosphate.

9. The immunogenic composition of claim 7, wherein the C. difficile Cwp2 protein comprises a sequence as set forth in SEQ ID NO: 18 or a sequence having at least 95%, 98%, 99% or 99.8% identity thereto.

10. The immunogenic composition of claim 7, wherein the immunogenic composition elicits at least a B cell response, a CD4+ T cell response, including Th1, Th2, or Th17, or a CD8+ T cell response.

11. The immunogenic composition of claim 7, wherein the pharmaceutically acceptable carrier comprises a nanoparticle or a liposome.

12. The immunogenic composition of claim 7, wherein the immunogenic composition further comprises at least one other pharmaceutical product, wherein the at least one other pharmaceutical product is an antibiotic.

13. The immunogenic composition of claim 12, wherein the antibiotic is selected from one or more metronidazole, amoxycillin, tetracycline, erythromycin, clarithromycin or tinidazole.

14. A method of treating or preventing C. difficile infection in a subject, comprising:
administering to the subject a therapeutically effective dose of a vaccine, wherein the vaccine comprises an isolated protein or a vector, wherein the isolated protein or the vector comprises a C.difficile Cwp2 protein or a fragment thereof.

15. The method of claim 14, wherein the vaccine further comprises a pharmaceutically acceptable carrier, an adjuvant or a combination thereof, wherein the adjuvant comprises alum, aluminum hydroxide or aluminum phosphate, wherein the pharmaceutically acceptable carrier comprises a nanoparticle or a liposome.

16. The method of claim 14, wherein the vaccine is administered intravenously, intramuscularly, intraperitoneally, intradermally, or subcutaneously to the subject.

17. The method of claim 14, wherein the vector is a plasmid, an expression vector or a viral vector.

18. The method of claim 14, wherein C. difficile Cwp2 protein comprises a sequence as set forth in SEQ ID NO: 18, wherein the Cwp2 protein is encoded by a polynucleotide, wherein the polynucleotide comprises a sequence as set forth in SEQ ID NO: 1 or a sequence having at least 95%, 98%, 99% or 99.8% identity thereto.


SEQUENCE LISTING (18 sequences)

SEQ ID NO: 1 - DNA, 1872 bp, synthetic construct
  source 1..1872: mol_type = other DNA
  sig_peptide 1..84
  misc_feature 85..954: note = Cwp_A gene
  misc_feature 955..1872: note = transmembrane region
atgaataaaaaaaatctttctgtaattatggctgctgcaatgataagtacatcagtagctccagtttttgctgcagaaactacacaggtaaaaaaagaaacaataactaagaaagaagctacagaactagtttcgaaagttagagatttaatgtctcaaaagtatactggtggttctcaagttggacaaccaatatatgaaataaaagttggcgagactttatcaaaattaaaaataataactaatatagatgaattagagaaattagtaaatgctttgggagaaaataaagaacttattgtaactataacagataaagggcatataacaaatagtgcaaatgaagtagttgcagaagcaactgaaaaatatgaaaattcagcagacctttccgctgaggctaattctataacagaaaaagctaaaactgaaactaatggaatttataaagttgcagatgtaaaagcttcatatgatagtgctaaagataagttagttataactttaagagataaaacagacacagtaacttctaaaactatagagataggtattggtgatgaaaaaattgatttaacagcaaatccagttgattcaacgggaacaaacttagacccttctacagaaggatttagagtaaataaaatcgttaaactaggtgtagcaggagctaaaaatattgatgatgtccaattagctgaaataactataaaaaatagtgacctaaatacagtttcaccacaagatttatatgatggatatagattaactgttaaaggtaatatggtagcaaatggaacatcaaagtcaattagtgatatttcatcaaaagattcagaaacaggaaagtataaatttactattaagtatactgatgcatctggaaaagcaatagagcttactgtagaaagtactaatgaaaaagatttaaaagatgccaaagctgcattagaaggtaattcaaaggttaaattgatagctggagatgatagatatgcaactgcagtggctatagcaaaacaaacaaaatatactgacaatatagttatagttaattcaaataaactagttgatggattagcagctacaccacttgctcaatctaaaaaagcacctatattattagcatccgataatgaaataccaaaagtaactttagattatataaaagatataattaagaaaagcccatcagctaaaatatatatagtaggtggagaatcagcagtatcaaatacagctaaaaagcaattagaatcagtaactaagaatgttgaaagactagctggagatgatagacatatgacttctgtagcagtagcaaaagctatggggtcttttaaagatgcatttgtagtaggtgcgaaaggggaggctgatgctatgagtatagctgccaaagctgctgaacttaaggctcctataatagtaaatggctggaatgatctttcagcagacgctatcaaattgatggatggaaaagagattggtatagttggtggttctaacaatgtatctagtcaaattgaaaatcaacttgctgatgttgataaagatagaaaagttcaaagagttgaaggagaaacaagacacgatactaatgctaaggttatagaaacatattatggaaaattagataaactatatatagcaaaagatggatatggaaataatggtatgctagtagatgcattggcagcaggacctctagcagcaggtaaaggtccaatacttctagctaaagctgatataacagactcacaaaggaatgcacttagtaaaaaattaaatcttggtgcagaagtaactcaaataggtaatggagttgaattgacagtaatacaaaagatagctaaaatactaggttggtaa

SEQ ID NO: 2 - DNA, 870 bp, synthetic construct
  source 1..870: mol_type = other DNA
caggtaaaaaaagaaacaataactaagaaagaagctacagaactagtttcgaaagttagagatttaatgtctcaaaagtatactggtggttctcaagttggacaaccaatatatgaaataaaagttggcgagactttatcaaaattaaaaataataactaatatagatgaattagagaaattagtaaatgctttgggagaaaataaagaacttattgtaactataacagataaagggcatataacaaatagtgcaaatgaagtagttgcagaagcaactgaaaaatatgaaaattcagcagacctttccgctgaggctaattctataacagaaaaagctaaaactgaaactaatggaatttataaagttgcagatgtaaaagcttcatatgatagtgctaaagataagttagttataactttaagagataaaacagacacagtaacttctaaaactatagagataggtattggtgatgaaaaaattgatttaacagcaaatccagttgattcaacgggaacaaacttagacccttctacagaaggatttagagtaaataaaatcgttaaactaggtgtagcaggagctaaaaatattgatgatgtccaattagctgaaataactataaaaaatagtgacctaaatacagtttcaccacaagatttatatgatggatatagattaactgttaaaggtaatatggtagcaaatggaacatcaaagtcaattagtgatatttcatcaaaagattcagaaacaggaaagtataaatttactattaagtatactgatgcatctggaaaagcaatagagcttactgtagaaagtactaatgaaaaagatttaaaagatgccaaagctgcattagaa

SEQ ID NO: 3 - DNA, 33 bp, synthetic construct
  source 1..33: mol_type = other DNA
agatgctagccaggtaaaaaaagaaacaataac

SEQ ID NO: 4 - DNA, 33 bp, synthetic construct
  source 1..33: mol_type = other DNA
ggtgctcgagttattctaatgcagctttggcat

SEQ ID NO: 5 - DNA, 19 bp, synthetic construct
  source 1..19: mol_type = other DNA
tgcaccaccaactgcttag

SEQ ID NO: 6 - DNA, 19 bp, synthetic construct
  source 1..19: mol_type = other DNA
ggatgcagggatgatgttc

SEQ ID NO: 7 - DNA, 21 bp, synthetic construct
  source 1..21: mol_type = other DNA
aaagagataatctggctctgc

SEQ ID NO: 8 - DNA, 20 bp, synthetic construct
  source 1..20: mol_type = other DNA
gctctgagacaatgaacgct

SEQ ID NO: 9 - DNA, 21 bp, synthetic construct
  source 1..21: mol_type = other DNA
ccaccacgctcttctgtctac

SEQ ID NO: 10 - DNA, 20 bp, synthetic construct
  source 1..20: mol_type = other DNA
agggtctgggccatagaact

SEQ ID NO: 11 - DNA, 18 bp, synthetic construct
  source 1..18: mol_type = other DNA
ggagaaagcggataccaa

SEQ ID NO: 12 - DNA, 18 bp, synthetic construct
  source 1..18: mol_type = other DNA
tgtgaggactaccgagcc

SEQ ID NO: 13 - DNA, 25 bp, synthetic construct
  source 1..25: mol_type = other DNA
tctcgaatgtaccaggagccatatc

SEQ ID NO: 14 - DNA, 22 bp, synthetic construct
  source 1..22: mol_type = other DNA
agcaccttggaagccctacaga

SEQ ID NO: 15 - DNA, 23 bp, synthetic construct
  source 1..23: mol_type = other DNA
agcacagtggtgaaagagacctt

SEQ ID NO: 16 - DNA, 22 bp, synthetic construct
  source 1..22: mol_type = other DNA
tccaatgcatagctggtgattt

SEQ ID NO: 17 - protein, 290 aa, synthetic construct
QVKKETITKKEATELVSKVRDLMSQKYTGGSQVGQPIYEIKVGETLSKLKIITNIDELEKLVNALGENKELIVTITDKGHITNSANEVVAEATEKYENSADLSAEANSITEKAKTETNGIYKVADVKASYDSAKDKLVITLRDKTDTVTSKTIEIGIGDEKIDLTANPVDSTGTNLDPSTEGFRVNKIVKLGVAGAKNIDDVQLAEITIKNSDLNTVSPQDLYDGYRLTVKGNMVANGTSKSISDISSKDSETGKYKFTIKYTDASGKAIELTVESTNEKDLKDAKAALE

SEQ ID NO: 18 - protein, 623 aa, synthetic construct
MNKKNLSVIMAAAMISTSVAPVFAAETTQVKKETITKKEATELVSKVRDLMSQKYTGGSQVGQPIYEIKVGETLSKLKIITNIDELEKLVNALGENKELIVTITDKGHITNSANEVVAEATEKYENSADLSAEANSITEKAKTETNGIYKVADVKASYDSAKDKLVITLRDKTDTVTSKTIEIGIGDEKIDLTANPVDSTGTNLDPSTEGFRVNKIVKLGVAGAKNIDDVQLAEITIKNSDLNTVSPQDLYDGYRLTVKGNMVANGTSKSISDISSKDSETGKYKFTIKYTDASGKAIELTVESTNEKDLKDAKAALEGNSKVKLIAGDDRYATAVAIAKQTKYTDNIVIVNSNKLVDGLAATPLAQSKKAPILLASDNEIPKVTLDYIKDIIKKSPSAKIYIVGGESAVSNTAKKQLESVTKNVERLAGDDRHMTSVAVAKAMGSFKDAFVVGAKGEADAMSIAAKAAELKAPIIVNGWNDLSADAIKLMDGKEIGIVGGSNNVSSQIENQLADVDKDRKVQRVEGETRHDTNAKVIETYYGKLDKLYIAKDGYGNNGMLVDALAAGPLAAGKGPILLAKADITDSQRNALSKKLNLGAEVTQIGNGVELTVIQKIAKILGW